(19) United States
(12) Patent Application Publication
(10) Pub. No.: US 2014/0154241 A1
(43) Pub. Date: Jun. 5, 2014

(54) BINDING PEPTIDES I

(71) Applicants: Duncan McGregor, Aberdeen (GB); William Eldridge, Aberdeen (GB); Simon Robins, Aberdeen (GB); Marie Fernie, Aberdeen (GB); Tricia White, Aberdeen (GB); Stuart Pritchard, Aberdeen (GB); Susan King, Aberdeen (GB)

(72) Inventors: Duncan McGregor, Aberdeen (GB); William Eldridge, Aberdeen (GB); Simon Robins, Aberdeen (GB); Marie Fernie, Aberdeen (GB); Tricia White, Aberdeen (GB); Stuart Pritchard, Aberdeen (GB); Susan King, Aberdeen (GB)

(73) Assignee: CYCLOGENIX LTD, Aberdeen (GB)

(21) Appl. No.: 13/991,178

(22) PCT Filed: Dec. 5, 2011
(86) PCT No.: PCT/GB11/52401

§ 371 Date: Jan. 23, 2014

Related U.S. Application Data
(60) Provisional application No. 61/419,352, filed on Dec. 3, 2010.

(51) Int. Cl. C07K 16/18

(52) U.S. Cl. CPC C07K 16/18
USPC 424/133.1; 530/387.3; 530/391.7

(57) ABSTRACT

A modified igNAR peptide sequence derived from a wild-type igNAR peptide sequence is diversified by mutating the amino acid sequence at 50% or more of the amino acids in the CDR3 loop region and optionally at 50% or more of the amino acids in the CDR3 loop region. The modified igNAR peptide may have the sequence of SEQ ID NO: 8, 10 or 50 to 85. The modified igNAR peptides have binding activity against albumin protein sequences, such as human serum albumin. These modified igNAR peptides may have utility in extending the in vivo half-life of biological molecules e.g. therapeutic agents, and so may be used in medicine.

### FIELD OF THE INVENTION

This invention relates to modified igNAR peptides having desirable functions, such as binding affinity to target ligands, and also to igNAR peptide framework libraries for selecting such modified igNAR peptides. In particular, the invention relates to modified igNAR variable-domain peptides that bind to albumin protein, and their use in extending in vivo half-lives of biological molecules.

### BACKGROUND OF THE INVENTION

Most proteins are folded into defined three-dimensional structures, and the type of three-dimensional structure is often used to classify and identify the family of proteins to which a particular protein belongs. Often, the members of a protein family contain relatively conserved sequence regions that are responsible for the three-dimensional folding of the protein and thus determine its structure; and relatively less conserved or variable sequence regions (e.g. in loops or flexible elements of the protein) that may determine or fine tune the function and activity of the protein.

Some proteins (and families of proteins) have been subject to extensive engineering in order first to understand how they work, and second to produce altered activities and new uses. Certain classes of protein structure (or framework), such as antibodies and zinc fingers, have been found particularly useful for these types of engineering projects. Beneficially, only select regions of a protein are modified (engineered), so that desirable properties of the natural protein, such as three-dimensional folding and certain functionalities can be retained. The regions that are not modified (i.e. the constant regions) may, therefore, be considered to represent the natural protein's scaffold or framework. The regions, domains or loops in the protein that are less critical for the protein's structure can then be modified or randomised to alter the functionality of the wild-type protein and, hopefully, to obtain new and useful properties, such as binding affinity for a desirably target molecule.

The technique of using a protein “scaffold” and engineering of loops or regions within the scaffold to alter activity is perhaps most notable with regard to the field of antibodies and antibody fragments, which have a natural repertoire of variable regions or loops. The variable loops of antibodies have been extensively engineered to produce peptides having improved binding (e.g. affinity and/or specificity) to known ligands, and also to expand the binding substrates for particular antibody frameworks (see for example, Knappik et al., (2000), J. Mol. Biol., 296, 57-86; and EP 1025218). The engineering of non-antibody frameworks has also been reviewed, for example, by Hosse et al., (2006), Protein Sci., 15, 14-27.

The shark immunoglobulin super-family protein, which is known as the immunoglobulin New Antigen Receptor (igNAR), was originally isolated and identified from the nurse shark, Ginglymostoma cirratum, in 1995 (Greenberg et al., (1995), Nature, 374, 168-173). IgNAR proteins have some structural similarities to mammalian antibody/immunoglobulin proteins. Indeed, analysis of the mutation patterns in different forms of igNAR has suggested that it is one of the immunoglobulin species responsible for the adaptive immune response in sharks, i.e. it appears to undergo hypermutation and affinity maturation as an antigen-driven process, similar to that observed in human and murine immunoglobulins.

The mature igNAR consists of two protein chains each with one variable domain and (generally) five constant domains. Detailed analysis has revealed the existence of two igNAR types in the Nurse shark, Type I and Type II. In some shark species, such as the Wobbegong shark, only the Type II igNAR has been identified. Type I proteins contain an additional framework disulphide bridge that is absent from Type II proteins. Both types possess long CDR3 loops in the variable domain and, like camelid VHH antibodies, the stability and conformation of these loops appears to be maintained by additional disulphide bridges.

Flajnikand and co-workers demonstrated that the primary NAR response is through hypermutation of the CDR3 loop, followed by affinity maturation of the CDR1 region (Greenberg et al., 1995). The Wobbegong igNAR protein framework has previously been used as a scaffold for the selection of new functionalities from a CDR3 peptide library (Nuttall et al. (2001), Mol. Immunol. 38, 313-326).

The use of naturally occurring single domain proteins as scaffolds for the building of libraries and the isolation of binding proteins having desirable new functionalities may offer a number of advantages over traditional antibody engineering. For example, the removal of the hydrophobic interfaces, linkers, and constant domains may enhance protein expression, stability, and even therapeutic activity, e.g. tumour penetration. The igNAR proteins thus appear to represent a functional single domain molecule, remarkably similar in structure to the camelid VHH antibodies, but distinct at the sequence level.

However, a known problem with many therapeutic molecules, in particular biologicals (such as peptide or polypeptide drugs, polynucleotides, etc.), is their short half-life when exposed to in vivo physiological conditions, for example, in the mammalian gut or circulatory systems. This problem often necessitates the administration of such therapeutics at higher frequency and/or higher concentration than would otherwise be necessary to maintain desirable systemic concentrations of the drug. Another approach is the use of sustained release formulations in order to maintain the serum levels necessary for therapeutic effects. Frequent systemic administration of drugs is associated with considerable negative side effects. For example, frequent (e.g. daily) systemic injections represent a considerable discomfort to the subject, and pose a high risk of administration related infections. Further, it may require hospitalisation or frequent visits to the hospital, in particular when the therapeutic is to be administered intravenously. Moreover, in long-term treatments, daily intravenous injections can also lead to considerable side effects of tissue scarring and vascular pathologies caused by the repeated puncturing of vessels. Similar problems are known for all frequent systemic administrations of therapeutics, like for example, the administration of insulin to diabetics, or interferon drugs to patients suffering from multiple sclerosis. All these factors lead to a decreased patient compliance and increased costs for the health system.

Therefore, it would be desirable to be able to increase the in vivo (e.g. serum) half-life of therapeutics in mammals.

Accordingly, the present invention seeks to overcome or at least alleviate one or more of the problems in the prior art.

### SUMMARY OF THE INVENTION

In general terms, the present invention provides a modified igNAR peptide or protein sequence that has new and useful properties, such as binding affinity for a target peptide sequence. A suitable target peptide comprises an albumin, such as a human albumin and particularly human serum albumin. More specifically, the invention relates to amino acid sequences derived from Wobbegong igNAR protein. Albumin-binding igNARs may have value in extending the in vivo half-life of therapeutic molecules that can be linked to the albumin-binding igNAR sequence. In addition, the invention relates to compositions comprising such modified igNAR peptides and to therapeutic and diagnostic molecules and compositions comprising such modified igNAR peptides. The invention may further relate to modified igNAR protein frameworks or scaffolds which can be used for the selection of de novo binding domains having desired binding characteristics, such as affinity for new target molecules and/or high affinity for known or new ligands. Furthermore, the invention may relate to methods for the selection of modified igNAR peptides that have one or more desirable activity, such as binding affinity for new target molecules/ligands, such as peptide sequences.

Thus, in a first aspect, the invention provides a modified igNAR peptide sequence derived from a wild-type igNAR peptide sequence, which is diversified by mutating the amino acid sequence at 50% or more of the amino acids in the CDR3 loop region. In some embodiments, the diversified CDR3 loop region of the modified igNAR peptide may comprise a greater or lesser number of amino acids than the wild-type CDR3 loop. For example, irrespective of the number of amino acid residues in the wild-type CDR3 loop, the modified CDR3 loop may consist of between 6 and 30 amino acids or between 10 and 20 amino acids. In one embodiment, the modified CDR3 loop consists of between 11 and 18 amino acids. In some specific embodiments, the modified CDR3 loop consists of 11, 13, 16 or 18 amino acids. A particularly suitable CDR3 loop sequence has 16 amino acids. In particularly suitable embodiments less than 50%, less than 20% or less than 10% of the wild-type residues in CDR3 of a wild-type igNAR protein are retained in the modified igNAR of the invention. Where the modified igNAR peptide or protein contains one or more cysteine residues in its CDR3 loop sequence, beneficially the cysteine is not in the same relative position to that of any cysteine residues in wild-type sequence.

In one embodiment, the modified igNAR peptide is derived from Wobbegong shark igNAR peptide or a fragment thereof. In this embodiment the CDR3 loop is represented by the peptide sequence at positions 85 to 97 of SEQ ID NO: 86 (and/or any amino acids inserted, deleted or substituted within this region of the igNAR variable domain. A fragment of a modified igNAR peptide or protein sequence may be a fragment of a wild-type igNAR variable domain peptide comprising at least 60, at least 70, at least 80, at least 90 or at least 100 contiguous amino acids from the wild-type variable domain sequence from which it was derived. The modified igNAR peptide sequence may comprise a sequence having at least 90%, at least 95% or at least 98% identity to the amino acid sequence at positions 1 to 84 and 101 to 110 of SEQ ID NO: 8 or of SEQ ID NO: 10. In another embodiment, the modified igNAR peptide sequence comprises a sequence having at least 90%, at least 95% or at least 98% (e.g. 99% or 100%) identity to the amino acid sequence of SEQ ID NO: 8 or of SEQ ID NO: 10. In yet another embodiment, the modified igNAR peptide sequence, or an igNAR protein, variable domain fragment or heavy chain antibody of the invention may comprise a sequence having at least 60%, at least 70%, at least 80%, or at least 90% identity to the amino acid sequence of SEQ ID NO: 9; most suitably, the sequence is found in the CDR3 loop region. In one beneficial embodiment, the modified igNAR peptide or protein sequence of the invention comprises the amino acid sequence of SEQ ID NO: 9; and in a particularly advantageous embodiment, the modified igNAR peptide sequence comprises the amino acid sequence of SEQ ID NO: 8 or of SEQ ID NO: 10. Furthermore, the modified igNAR peptide of the invention may comprise at least 50, at least 60, at least 70, at least 80, at least 90 or at least 100 amino acids of the parent igNAR protein sequence from which it is derived. Suitably, the peptide fragment of the igNAR protein or antibody of the invention has binding activity to a desired target ligand.

The modified igNAR peptides of the invention may bind to albumin proteins or peptide sequences. Target albumins are suitably of mammalian origin, such as from mouse, rat, pig or primate. Advantageously, the albumin sequence is a human albumin sequence. Human albumins that may be bound by the modified igNAR peptides of the invention include human serum albumin (HSA).

The modified igNAR peptide sequence of the invention may be further diversified in order to improve or fine tune its binding activity for the target molecule, such as albumin. Further diversification of the wild-type peptide sequence from which the modified igNAR of the invention is derived may help to improve binding specificity, selectivity and/or affinity to the target molecule. In one embodiment the further diversifications are introduced into the CDR1 loop sequence (amino acids 19 to 25 of SEQ ID NO: 86) of the igNAR variable domain. Accordingly, the modified igNAR peptide of the invention may comprise mutations in the amino acid sequence at 50% or more of the amino acids in the CDR1 loop region. Thus, the modified igNAR peptide sequence may comprise 50% or less, 30% or less, or 10% or less identity to the amino acid sequence of the wild-type CDR1 (SEQ ID NO: 11) in SEQ ID NO: 86.

Preferred modified igNAR variable domain peptides therefore comprise diversifications in the CDR1 and CDR3 loop regions of a wild-type igNAR peptide, such as SEQ ID NO: 86. Thus, the invention encompasses modified igNAR peptides comprising the sequence of any of SEQ ID NOs: 51 to 85. The invention further encompasses modified igNAR peptides having at least 90%, at least 95% or at least 98% identity to the amino acid sequence of any of SEQ ID NO: 51 to 85. In some embodiments, the modified igNAR peptides comprise fragments of the full-length peptide sequences of the invention, such fragments may comprise at least 70, at least 80, at least 90 or at least 100 contiguous amino acids of SEQ ID NOs: 51 to 85, and fragments having at least 90%, at least 95% or at least 98% identity thereto. Suitably the modified igNAR peptide of the invention is a fragment of an igNAR protein, such as an igNAR variable domain. In some embodiments, the modified igNAR peptide comprises a heavy chain antibody or fragment thereof.

Beneficially, the modified igNAR peptide, igNAR antibody or antibody fragment of the invention binds an albumin target molecule with a dissociation constant (Kd) of less than 10 μM, less than 1 μM, or less than 100 pM.

It will be appreciated that modified igNAR proteins of the invention, including heavy chain antibodies and fragments thereof, may be further derivatised or conjugated to additional molecules and that such derivatives and conjugates fall within the scope of the invention. Beneficially, the modified igNAR peptide sequence of the invention is conjugated, fused, linked or otherwise associated with another moiety. The other moiety may be another igNAR peptide sequence or may be a non-igNAR moiety. Advantageously, the moiety is a biological molecule (such as a polynucleic acid or polypeptide); and preferably a therapeutic molecule or agent. The non-igNAR moiety may be an antibody molecule or fragment thereof.

In accordance with another aspect of the invention, the modified igNAR peptide sequence may be used to extend the half-life of a biological molecule in vivo, for example, in a mammal such as a human.

Modified igNAR peptides and antibodies or fragments thereof of the invention may be further modified to provide increased stability for therapeutic and other in vivo applications.

In another aspect there is provided a nucleic acid sequence encoding the modified igNAR peptide sequence, antibody, or antibody fragment of the invention. The nucleic acid may comprise a vector sequence, such as an expression vector or construct comprising the nucleic acid of the invention.

The peptide, protein or nucleic acid of the invention may be for use in medicine. For example, the use may be for treating a disease or condition in an individual, such as cancer, a neurodegenerative disease or a diabetic condition. Accordingly, the invention encompasses therapeutic and diagnostic uses for the modified igNAR proteins/peptides of the invention. Aspects and embodiments of the invention therefore include formulations, medicaments and pharmaceutical compositions comprising the modified igNAR proteins or nucleic acids of the invention.

In one aspect the invention relates to a method of treating, preventing or alleviating a disease in a mammal, the method comprising administering to a subject in need thereof a therapeutically effective amount of the modified igNAR peptide or antibody/antibody fragment of the invention. In particular, the modified igNAR protein or peptide sequence may be conjugated, fused, linked or otherwise associated with a therapeutic biological molecule, e.g. as a fusion protein comprising a biologically active agent. Thus, in a beneficial embodiment the proteins and peptides of the invention are for use in extending the in vivo (e.g. serum) half-life of a therapeutic biological molecule by binding to an albumin protein, such as a human serum albumin. The modified igNAR proteins and particularly fusion proteins of the invention may be used in the treatment of various diseases and conditions of the human or animal body. The therapeutic fusion protein/complexes of the invention are particularly beneficial for use in the treatment of diseases requiring frequent and/or long-term and/or repetitive administrations of the therapeutic molecule, and/or where the therapeutic molecule is susceptible to degradation or has a relatively short half-life in vivo. Suitable diseases or conditions include cancers, neurodegenerative diseases and diabetic disorders. Treatment may also include preventative as well as therapeutic treatments and alleviation of a disease or condition.

In yet another aspect there is provided a method of extending the in vivo or serum half-life of a molecule, preferably a therapeutic agent, by conjugating the molecule to a modified igNAR peptide capable of binding to an albumin. The igNAR peptide, antibody or fragment may be an albumin-binding igNAR peptide according to the invention. The molecule and the igNAR peptide may be joined or associated with each other in any suitable manner, as described elsewhere herein.

The invention may also relate to a naïve igNAR variable domain protein library which has a consensus amino acid sequence derived from a wild-type igNAR variable domain protein sequence (SEQ ID NO: 86), wherein the amino acid sequence encoding the CDR3 loop has the sequence X₆ to X₃₀, where X is any amino acid, the numbers in subscript indicates the number of amino acids (SEQ ID NO: 88). Suitably less than 50%, less than 20% or less than 10% of the amino acids at each X position are wild-type. The invention may more suitably relate to a naïve igNAR protein library which has a consensus amino acid sequence derived from a wild-type igNAR protein sequence, wherein the amino acid sequence encoding the CDR3 loop has the sequence X₁₁, X₁₃, X₁₆ or X₁₈, where X is any amino acid, the numbers in subscript indicates the number of amino acids; and suitably wherein less than 50%, less than 20% or less than 10% of the amino acids at each X position are wild-type. Naive igNAR peptide libraries of the invention include the sequences of SEQ ID NOs: 89 to 92. Advantageously, the naïve igNAR protein library is derived from the Wobbegong shark. The naïve igNAR protein library may comprise an amino acid sequence having at least 80%, at least 90%, at least 98% or 100% identity to the amino acids at positions 1 to 84 and 101 to 110 of SEQ ID NO: 8 or of SEQ ID NO: 10; and wherein the sequence between the amino acids at positions 84 and 101 (i.e. from Glu85 to His100 in SEQ ID NO: 8 or SEQ ID NO: 10, respectively) is the sequence X₁₁, X₁₃, X₁₆ or X₁₈, where X is any amino acid, and the numbers in subscript indicate the number of amino acids. Suitably, each X position is randomly selected from one or at least 2, at least 4, at least 10 or all 20 of the naturally occurring amino acids. Advantageously, the naïve igNAR protein library of the invention may additionally include a second region of diversification in the CDR1 loop peptide sequence. Accordingly, the amino acid sequence encoding the CDR1 loop region may be diversified at one or more positions, and may suitably be diversified at all positions of the CDR1 sequence. A pool of nucleic acid molecules encoding at least one igNAR protein of the naïve igNAR protein library is also encompassed.

The naïve igNAR protein libraries of the invention are beneficially used in the selection of a modified igNAR protein to bind a target ligand. Suitably, the target ligand is an albumin peptide sequence and most suitably a human (serum) albumin. The naïve igNAR peptide library of the invention may be used in a method for selecting useful modified igNAR peptide sequences. Suitably, the naïve igNAR peptide library is expressed on the surface of phage particles (e.g. in a phage display procedure) in order to select for useful modified igNAR peptide sequences. Selected modified igNAR peptides of the invention may be isolated and optionally derivatised and/or conjugated to another moiety, such as a non-igNAR peptide moiety. For example, the modified igNAR peptides of the naïve igNAR peptide library may be conjugated, fused, linked or otherwise associated with a moiety such as a therapeutic molecule. The conjugated/associated igNAR and therapeutic molecule may be administered to a mammal, such as a mouse, rat, pig, primate or human to select or identify modified igNAR peptides having desirable properties in vivo.

It should also be appreciated that, unless otherwise stated, optional features of one or more aspects of the invention may be incorporated into any other aspect of the invention. All references cited herein are incorporated by reference in their entirety. Unless otherwise defined, all technical and scientific terms used herein have the same meaning as commonly understood by one of ordinary skill in the art to which this invention belongs.

### BRIEF DESCRIPTION OF THE DRAWINGS

The invention is further illustrated by the accompanying drawings in which:

FIG. 1 illustrates the wild-type (parental) Wobbegong shark igNAR variable domain protein scaffold sequence (SEQ ID NO: 86) as synthesised by GeneArt. The IgNAR DNA sequence is underlined and flanking vector DNA sequences are indicated in italics. The wild-type CDR3 amino acid loop sequence which was replaced in the libraries of the invention is underlined in the peptide sequence (CDR3 loop region between Tyr85 and Lys97). Additionally, the CDR1 region between amino acid positions 19 and 25 (particular residues Ile19 to Asn20 and Val22 to Asp25) is indicated by a double underline in the peptide sequence. An invariant cysteine residue at position 21 is flanked on either side by the variable CDR1 region.

FIG. 2 is a schematic illustration of the Wobbegong igNAR peptide primary library (row (a)). The fixed cysteine residue (marked “C”) at the beginning of the 13 amino acid CDR3 loop sequence is illustrated. CDR3 loop libraries of the invention contained variable length CDR3 regions of 11, 13, 16 or 18 residues. Libraries having 11, 13 and 18 amino acids contain a second fixed position cysteine residue (C) at the wild-type position (row (b)) or at a different position within CDR3 (row (c)). The 16-amino acid length CDR3 library had no fixed cysteine residue within the CDR3 loop sequence and so had to rely of randomly encoded cysteine residues.

FIG. 3 depicts the pSP1 phagemid vector multiple cloning site. Mutant Wobbegong igNAR library DNA constructs were cloned as NcoI-NotI fragments, in-frame with full-length pIII, separated by a short linker and a supE TAG codon. The pelB leader sequence and the beginning of the pIII gene are also indicated.

FIG. 4 illustrates the results of an ELISA screen for HSA binding proteins from the primary CDR3 libraries. (A) Multiple albumin-binding peptides were isolated in the output of round 3 of the library selection (binding strength, z-axis; selected igNAR peptide identifier, x-axis and y-axis coordinates). (B) Background binding strength of the selected peptides shown in FIG. 4A in ELISA assays against β-galactosidase coated wells (binding strength, z-axis; selected igNAR peptide identifier, x-axis and y-axis coordinates).

FIG. 5 shows the results of an ELISA assay to illustrate the binding specificity of a selected albumin-binding clone. Albumin-binding clone A11 from the ELISA plate illustrated in FIGS. 4A and 4B was tested for binding strength against human (HuSA), mouse (MuSA) and rat (RatSA) serum albumin, as well as to non-target proteins: trkA-Fc fusion protein (TrkA) and β-galactosidase (b-gal). As controls, peptides previously selected to bind to trkA and β-galactosidase were used.

### DETAILED DESCRIPTION OF THE INVENTION

In order to assist with the understanding of the invention several terms are defined herein.

The term “peptide” as used herein (e.g. in the context of a modified igNAR peptide or framework) refers to a plurality of amino acids joined together in a linear or circular chain. The term oligopeptide is typically used to describe peptides having between 2 and about 50 or more amino acids. Peptides larger than about 50 are often referred to as polypeptides or proteins. For purposes of the present invention, the term “peptide” is not limited to any particular number of joined amino acids, and the term “peptide” is thus used interchangeably with the terms “oligopeptide”, “polypeptide” and “protein”. Suitably, a modified igNAR peptide of the invention contains between about 100 and about 125 amino acid residues. However, igNAR fusion proteins may contain any number of amino acids. Furthermore, the invention also provides peptide fragments of full-length igNAR proteins, which have binding activity to desired target molecules. Such fragments comprise the CDR3 loop region and beneficially also the CDR1 loop region. An igNAR peptide fragment of the invention may, therefore, comprise at least 60 contiguous amino acids from SEQ ID NO: 8, or modified sequences thereof (e.g. sequences having at least 80%, at least 90%, at least 95% or at least 98% identity thereto). Suitably, an igNAR peptide fragment comprises at least 70, at least 80, at least 90 or at least 100 contiguous amino acids from SEQ ID NO: 8, or modified sequences thereof.

The term “amino acid” in the context of the present invention is used in its broadest sense and is meant to include naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

A “modified igNAR peptide” (or protein) of the invention is based on a wild-type igNAR protein that has been mutated (e.g. by amino acid substitution, deletion, addition) in at least one position. Thus, the modified igNAR peptide is conveniently derived from a wild-type igNAR protein or peptide sequence. In one beneficial embodiment it is derived from a variable domain peptide sequence of an igNAR protein. More suitably, it is derived from the wild-type igNAR protein of Wobbegong shark or a fragment thereof. Furthermore, it should be appreciated that, depending on the application, the modified igNAR peptide of the invention may comprise an additional peptide sequence or sequences at the N- and/or C-terminus in comparison to the corresponding wild-type (variable domain) peptide sequence from which it is derived: for example, additional short peptide sequences may be appended at the N-terminus for ease of protein expression and/or nucleic acid cloning. This is particularly convenient when the peptide is derived from a fragment, such as the variable domain of a larger wild-type protein sequence. Alternatively, the igNAR peptides of the invention may be fused to other peptide or non-peptide moieties. Such modified igNAR peptides are encompassed within the scope of the invention.

Modified igNAR peptides of the invention typically contain naturally occurring amino acid residues, but in some cases non-naturally occurring amino acid residues may also be present. Therefore, so-called “peptide mimetics” and “peptide analogues”, which may include non-amino acid chemical structures that mimic the structure of a particular amino acid or peptide, may also be used within the context of the invention. Such mimetics or analogues are characterised generally as exhibiting similar physical characteristics such as size, charge or hydrophobicity, and the appropriate spatial orientation that is found in their natural peptide counterparts. A specific example of a peptide mimetic compound is a compound in which the amide bond between one or more of the amino acids is replaced by, for example, a carbon-carbon bond or other non-amide bond, as is well known in the art (see, for example Sawyer, in Peptide Based Drug Design, pp. 378-422, ACS, Washington D.C. 1995). Such modifications may be particularly advantageous for increasing the stability of modified igNAR peptides and/or for improving or modifying solubility, bioavailability and delivery characteristics (e.g. for in vivo applications).

One aspect of the present invention is directed towards an igNAR peptide framework, scaffold or template (which terms are used interchangeably herein), which can be used to create libraries of modified igNAR peptides for screening to identify modified igNAR peptides having desirable physical properties and characteristics. It will be understood that the igNAR library framework may be a nucleic acid sequence or a peptide sequence. The igNAR framework of the invention may be derived from any suitable wild-type igNAR protein sequence, and is suitably derived from a fragment of an igNAR protein—typically the variable domain sequence or fragment thereof. Thus, it comprises a (conserved) core or backbone of amino acid residues of the wild-type peptide from which it is derived, with a plurality of amino acid mutations (e.g. substitutions) at various positions in comparison to the corresponding wild-type sequence. Conveniently, therefore, an “igNAR peptide framework” as used herein encompasses a library (or population) of different but related igNAR peptides based around a common core sequence with specific or random mutations at one or more positions within the domain; and as such may also be termed a igNAR peptide (or nucleic acid) framework library. Such a library having a mixture of peptides or nucleic acids that has not been optimised or selected to have a particular functionality is termed herein a “naïve” library. An individual peptide expressed from an igNAR peptide framework library and which does not have a wild-type sequence may also be considered to be a “modified igNAR peptide”, and its encoding nucleic acid can be considered a “modified igNAR nucleic acid”. Beneficially, a modified igNAR peptide of the invention adopts the characteristic three-dimensional folding pattern comprising 5 constant domains and 1 variable domain with peptide loop sequences (e.g. CDR1 and CDR3). In one beneficial embodiment, the igNAR framework is homologous (e.g. identical) to the wild-type protein sequence on which it is based, except for one or more amino acids in the CDR3 and/or CDR1 loop regions. However, in some cases, point mutations at defined positions in the wild-type protein sequence outside of the CDR1 and CDR3 regions may be made and tolerated, such that a functional and useful modified igNAR protein, antibody or variable domain fragment is achieved.

Any desirable ligand may be recognised (i.e. bound) by modified igNAR peptides of the invention, such as nucleic acids (e.g. DNA or RNA), small organic or inorganic molecules, proteins or peptides. A suitable ligand is a protein, and a particularly suitable ligand is a peptide sequence or “epitope” of a protein. A preferred target ligand is an albumin peptide sequence or protein.

Another aspect of the present invention is directed towards the identification and characterisation of modified igNAR peptides having a desired property, from amongst a population (or library) of mutant igNAR peptides based on an igNAR peptide framework. The library comprises a plurality of nucleic acid sequences (e.g. at least 10⁶, 10⁸, 10⁹, 10¹² or more different coding sequences) that can be expressed and screened to identify modified igNAR peptides having the desired property.

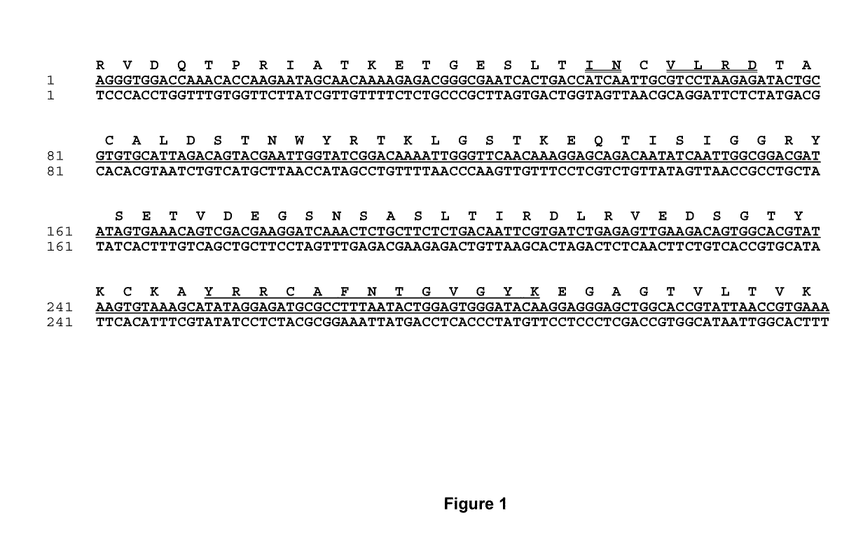

Typically, the modified igNAR peptide framework is derived from the Wobbegong shark type II igNAR variable domain protein sequence (SEQ ID NO: 86; FIG. 1). The modified igNAR peptide of the invention may thus be selected from a library of mutant Wobbegong igNAR variable domain protein sequences. A selected modified igNAR peptide of the invention may contain 5 or more, 7 or more, 10 or more, or 15 or more mutations relative to the wild-type igNAR variable domain protein sequence from which it is derived. A preferred form of mutation is an amino acid substitution. However, it is beneficial that the modified igNAR peptide be at least 70%, or at least 80%, or at least 90% identical to the corresponding variable domain wild-type sequence, so that the three-dimensional structure or fold of the functional variable domain is substantially maintained. Advantageously, the modified igNAR peptide of the invention comprises at least 1 cysteine residue within the CDR3 sequence (e.g. between positions 84 and 98 of the wild-type Wobbegong igNAR sequence shown in FIG. 1). Suitably the cysteine residue is located in a different position in the loop to that of any cysteine residue is the corresponding wild-type loop sequence. Furthermore, the modified igNAR peptide of the invention beneficially comprises at least 1 cysteine residue in the middle of/within the CDR1 sequence (e.g. from positions 19 and 25 of the wild-type Wobbegong igNAR sequence shown in FIG. 1).

By “derived from” it is meant that the peptide concerned includes one or more mutations in comparison to the primary amino acid sequence of the peptide on which it is based. Thus a modified igNAR peptide of the invention is considered to be derived from a wild-type protein/peptide sequence, such as from Wobbegong igNAR. Similarly, by “derivative” of a modified igNAR peptide it is meant a peptide sequence that has the selected, desired activity (e.g. binding affinity for a selected target ligand), but that further includes one or more mutations or modifications to the primary amino acid sequence of a modified igNAR peptide first identified by the methods of the invention. Thus, a derivative of a modified igNAR peptide of the invention may have one or more (e.g. 1, 2, 3, 4, 5 or more) chemically modified amino acid side chains compared to the modified igNAR from which it is derived. Suitable modifications may include pegylation, sialylation and glycosylation. In addition or alternatively, a derivative of a modified igNAR peptide may contain one or more (e.g. 1, 2, 3, 4, 5 or more) amino acid mutations, substitutions or deletions to the primary sequence of a selected modified igNAR peptide. Accordingly, the invention encompasses the results of maturation experiments conducted on a modified igNAR peptide to improve or alter one or more characteristics of the initially identified peptide. By way of example, one or more amino acid residues of a selected modified igNAR peptide sequence may be randomly or specifically mutated (or substituted) using procedures known in the art (e.g. by modifying the encoding DNA or RNA sequence). The resultant library or population of derivatised peptides may be selected—by any known method in the art—according to predetermined requirements: such as improved specificity against particular target ligands; or improved drug properties (e.g. solubility, bioavailability, immunogenicity etc.). Peptides selected to exhibit such additional or improved characteristics and that display the activity for which the modified igNAR peptide was initially selected may be considered to be derivatives of the modified igNAR peptide and fall within the scope of the invention. By way of example, where the modified igNAR peptide was first derived by mutating the wild-type amino acid sequence in the region of the CDR3 loop; a derivative of the modified igNAR peptide may be generated by then mutating the wild-type amino acid sequence in the region of the CDR1 loop so as to improve or modify the binding or activity profile of the first modified igNAR peptide.

In some cases it may be desirable to conjugate a modified or derivatised igNAR peptide of the invention to one or more additional modified igNAR peptides or fragments thereof in order to create a multimer, such as a dimer or trimer, of modified igNAR peptides—for example, to bind more than one target molecule simultaneously. Particularly preferred are dimers of modified igNAR peptide variable domain sequences of the invention. The target molecules may be either on the same or different molecules and may be the same or different, depending on requirements. Furthermore, the modified igNAR peptide of the invention may be conjugated to a non-igNAR peptide moiety. The term “conjugate” is used in its broadest sense to encompass all methods of attachment or joining that are known in the art. For example, the non-igNAR peptide moiety can be an amino acid extension of the C- or N-terminus of the modified igNAR peptide. In addition, a short amino acid linker sequence may lie between the modified igNAR peptide and the non-igNAR peptide moiety. The invention further provides for molecules where the modified igNAR peptide is linked, e.g. by chemical conjugation to the non-igNAR peptide moiety optionally via a linker sequence. Typically, the modified igNAR peptide will be linked to the other moiety via sites that do not interfere with the activity of either moiety. The term “conjugated” is used interchangeably with terms such as “linked”, “bound”, “associated”, “fused” or “attached”. A wide range of covalent and non-covalent forms of conjugation are known to the person of skill in the art, and fall within the scope of the invention. For example, disulphide bonds, chemical linkages and peptide chains are all forms of covalent linkages. Where a non-covalent means of conjugation is preferred, the means of attachment may be, for example, a biotin-(strept)avidin link or the like. Antibody (or antibody fragment)-antigen interactions may also be suitably employed to conjugate a modified igNAR peptide of the invention to another moiety, such as a non-igNAR peptide moiety.

A “non-igNAR peptide moiety” as used herein, refers to an entity that does not contain an igNAR peptide sequence or three-dimensional fold. The person of skill in the art understands and can determine whether a polypeptide molecule is an igNAR protein or peptide sequence, for example, by way of sequence homology or structure prediction or determination. Such non-igNAR peptide moieties include nucleic acids and other polymers, peptides, proteins, peptide nucleic acids (PNAs), antibodies, antibody fragments, and small molecules, amongst others. Suitably, a non-igNAR peptide moiety is a biological molecule (e.g. comprising a polynucleotide or peptide), and advantageously is a therapeutic or targeting molecule.

### IgNAR Peptides, Frameworks and Libraries

The shark immunoglobulin superfamily protein, termed the immunoglobulin New Antigen Receptor (igNAR), was originally identified from the nurse shark, Ginglymostoma cirratum, in 1995 (Greenberg et al., (1995), Nature, 374, 168-173). Mature igNAR consists of two protein chains each having one variable and five constant domains. It has been found to exist in both cell-bound and secretory forms. Although igNAR proteins have some structural similarities to mammalian antibody/immunoglobulin proteins, they lack the “light” immunoglobulin chains of typical antibodies. Thus, immune electronmicroscopy has revealed that the variable domains are free in solution and do not interact across an antibody V_{H}/V_{L}-type interface. Notably, therefore, the residues that would have been predicted to lie within such an interface are charged (polar) rather than hydrophobic, suggesting adaptation to a solvated environment. While the igNAR variable domain has extremely low sequence conservation with other immunoglobulin superfamily member variable domains (early phylogenetic studies placed the igNAR V-domain equidistant between antibodies and T-cell receptors), there is sufficient similarity to identify the framework and CDR regions of igNAR proteins. The closest matches to the Wobbegong shark type II igNAR variable domain sequence, excluding CDR3, are human lambda light chain (47%), rat T-cell receptor alpha (47%), lama V_{H} chain (47%) and zebra fish T-cell receptor alpha chain (47%). The CDR1 and CDR3 regions of the variable domain of igNAR proteins are typically highly variable—lacking sequence conservation between species; and the CDR3 region can be variable in length.

Analysis has also revealed the existence of two igNAR types in the Nurse shark: type I and type II, as previously mentioned. Both types possess long CDR3 loops and, like camelid VHH antibodies, the stability and conformation of these loops appears to be maintained by additional disulphide bridges. For Type I igNAR proteins there is a preponderance of paired cysteine residues within the CDR3 loop, suggesting the formation of intra-loop disulphide bridges. In contrast, a high proportion of Type II igNAR proteins possess paired cysteines in the CDR1 and CDR3 loops, which suggests the formation of inter-loop disulphide bridges.

The fact that igNAR lacks the additional light chains of conventional antibody molecules may be a particular benefit in the use of modified igNAR (variable domain) peptides for binding to desired target molecules. For example, the use of naturally occurring single domain proteins as scaffolds for the building of libraries and the isolation of binding proteins may have advantages over traditional antibody strategies. Furthermore, the removal of the hydrophobic interfaces, linkers, and constant domains may help to enhance protein expression, stability, and therapeutic activity (e.g. tumour penetration). Thus, an igNAR variable domain peptide appears to represent a functional single domain molecule, remarkably similar in structure to the camelid VHH antibodies, but distinct at the sequence level.

In the present invention, we have created modified igNAR variable domain peptides capable of binding to selected target molecules with desirable affinity and specificity. We have also generated igNAR peptide frameworks suitable for the generation of libraries of modified igNAR peptides, which can be screened for desirable properties, such as binding affinity to a chosen target ligand.

There are a number of igNAR proteins known in the art, and any of these may be suitable for use as igNAR peptide frameworks for the selection and synthesis of modified igNAR peptides as novel binding modules (as described herein). Thus, suitable igNAR proteins for use in accordance with the invention include polypeptides comprising the igNAR peptide sequences of any elasmobranch species, such as nurse or Wobbegong sharks.

In one embodiment the igNAR variable domain peptide or peptide framework is based on the wild-type Wobbegong igNAR peptide sequence displayed in FIG. 1, i.e. N′-RVDQTPRIATKETGESLTINCVLRDTACALDSTNWYRTKLGSTKEQTISIGGRYSETVDE GSNSASLTIRDLRVEDSGTYKCKAYRRCAFNTGVGYKEGAGTVLTVK-C′ (SEQ ID NO: 86); CDR1 region shown in italics; CDR3 loop sequence underlined; cysteine residues shown in bold), which is a type II igNAR protein. For the purposes of this invention, the numbering of the amino acid sequence of Wobbegong igNAR protein can be considered to begin with an N-terminal arginine residue (which is conveniently numbered as position 1), and end with a C-terminal lysine residue (which is conveniently numbered as position 107). However, it should be appreciated that different N-terminal and C-terminal residues have been reported and peptide sequences including such additional/alternative residues are incorporated within the scope of the invention. For example, a Wobbegong igNAR variable domain protein sequence has been reported to have an additional N-terminal alanine residue. In this case, the igNAR peptide sequence may be one amino acid longer than indicated in FIG. 1, and the residue numbers may then be adjusted by 1 to take the change into account. It should also be appreciated that in isolated modified igNAR peptides of the invention, the N-terminus may include amino acid sequences beneficial for protein expression or cloning. For ease of understanding the invention and for reason of internal consistency, the CDR3 loop of the wild-type Wobbegong igNAR protein is considered to begin at amino acid position 85 (i.e. Tyr) and end at amino acid position 97 (i.e. Lys) and is thus the 13 amino acid sequence, N′-YRRCAFNTGVGYK-C′ (SEQ ID NO: 87). However, the length and sequence of the CDR3 loop in different wild-type/natural igNAR proteins can vary considerably, and such diversity may be important for determining the epitope binding specificity of an igNAR protein.

Hence, the modified igNAR peptides and the igNAR peptide library frameworks of the invention have modified CDR3 loop regions, which are generated by way of amino acid deletion, insertion and/or diversification by mutation/substitution. The CDR3 loop in modified igNAR peptides of the invention can be any convenient length and sequence, depending on the target to be bound and any design criteria. For example, the sequence of the modified CDR3 loop may have between 6 and 30 amino acids and have any sequence. It may, therefore, be longer or shorter than the CDR3 sequence of the substantially wild-type protein framework into which it has been introduced. In some embodiments, the CDR3 loop sequences may have 11, 13, 16 or 18 amino acids. A particularly suitable modified igNAR peptide has a CDR3 loop of 16 amino acids. A preferred modified igNAR peptide comprises the CDR3 loop sequence of SEQ ID NO: 9. The modified igNAR peptide may comprise the amino acid sequence of SEQ ID NO: 8 or SEQ ID NO: 10. In peptide embodiments of the invention the modified igNAR peptide sequence may further comprise a mutation of Glu60 (see FIG. 1) to Lys.

In some embodiments the modified igNAR peptides and the igNAR peptide library frameworks of the invention may have modified CDR1 loop regions, which are generated by way of amino acid deletion, insertion and/or diversification by mutation/substitution—as for the modified CDR3 loop region above. The modified CDR1 loop may be the same or different length to the sequence of a wild-type igNAR protein CDR1 loop region. In a particularly suitable embodiment, the modified CDR1 loop is the same length as the CDR1 loop of the igNAR protein framework on which it is inserted. For example, when the modified igNAR peptide is based on Wobbegong igNAR the CDR1 loop is located at amino acid positions 19 to 25, i.e. the sequence N′-INCVLRD-C′ (SEQ ID NO: 11; see also FIG. 1). Beneficially, the modified CDR1 includes a cysteine residue; and preferably the cysteine residue is in the same position as the cysteine residue of the wild-type template.

A particularly suitable modified igNAR peptide has a CDR3 loop of 16 amino acids and a modified CDR1 loop sequence. A preferred modified igNAR peptide thus comprises the CDR3 loop sequence of SEQ ID NO: 9 and, in addition, a modified CDR1 loop sequence selected from one of SEQ ID NO: 16 to 50. More specifically, such a modified igNAR peptide may comprise an amino acid sequence having at least 80% and suitably at least 90% (e.g. at least 95% or at least 98%) amino acid identity to any of SEQ ID NOs: 8, 10 or 51 to 85. This embodiment thus may allow further specificity/affinity adjustment of the modified igNAR peptides through maturation of the CDR1 sequence in selected modified peptides of the invention. Accordingly, the igNAR (variable domain) peptide/protein could be considered a scaffold displaying a constrained two-loop library.

In igNAR variable domain peptide libraries of the invention, the amino acid residues at each of the diversified or mutated positions of the igNAR sequence from which a modified igNAR peptide is derived may be non-selectively randomised, i.e. by replacing each of the diversified/mutated amino acids with one of the other 19 naturally occurring amino acids; or may be selectively randomised, i.e. by replacing each of the specified amino acids with one from a defined sub-group of the remaining 19 naturally occurring amino acids. It will be appreciated that one convenient way of creating a library of mutant peptides with randomised amino acids at each selected location is to randomise the nucleic acid codon of the corresponding nucleic acid sequence that encodes the target amino acid. In this case, in any individual peptide expressed from the library, any of the 20 naturally occurring amino acids may be incorporated at the randomised position. Therefore, in some instances (e.g. approximately 5%), the wild-type amino acid residue may be incorporated by chance.

A suitable naïve igNAR variable domain peptide framework library of the invention may comprise the sequence: N′-RVDQTPRIATKETGESLTINCVLRDTACALDSTNWYRTKLGSTKEQTISIGGRYSETVDE GSNSASLTIRDLRVEDSGTYKCKA(X₆₋₃₀)EGAGTVLTVK-C′ (SEQ ID NO: 88); wherein X represents an amino acid which may be any of the 20 naturally-occurring amino acids, and the number is subscripts indicates the number of X amino acids in the modified sequence. In some embodiments, the modified sequence region denoted by X residues may have 11 to 18 amino acids; and more suitably may consist of 11, 13, 16 or 18 amino acids. In the corresponding nucleic acid molecule that encodes the peptide of SEQ ID NO: 88, X may be encoded by an NNK codon, wherein N represents an equal mixture of A, C, T and G, and K is an equal mix of G or T.

In some cases it may be beneficial to express igNAR peptides as a fusion protein, for example, to aid in the expression, screening or selection of desirable modified igNAR peptides. For example, the igNAR peptides, particularly library members, may be expressed with a linker sequence at the N- or C-terminus

While the above has been described primarily in relation to igNAR protein sequence framework derived from Wobbegong shark, it will be appreciated that other igNAR protein frameworks may alternatively be used, such as those from Nurse shark or other elasmobranch species (Roux et al., (1998), Proc. Natl. Acad. Sci. USA. 95, 11804-11809).

Albumin binding igNARs (in particular variable domain fragments of igNAR proteins) may have value in extending the in vivo half-life of therapeutic molecules linked to the albumin binding igNAR.

Expression and Characterisation of Peptides from Libraries

The modified igNAR peptides of the invention may conveniently be selected by screening libraries of peptides derived from an igNAR variable domain protein framework. The screening may be performed using any library generation and selection system known to the person of skill in the art, such as those identified below.

One approach is to produce a mixed population of candidate peptides by chemically synthesising a randomised library of e.g. 6 to 10 amino acid peptides (J. Eichler et al., (1995), Med. Res. Rev., 15, 481-496; K. Lam (1996) Anticancer Drug Des., 12, 145-167; and M. Lebl et al., (1997), Methods Enzymol., 289, 336-392). In another approach, candidate peptides are synthesised by cloning a randomised oligonucleotide library into an Ff filamentous phage gene, which allows peptides that are much larger in size to be expressed on the surface of the bacteriophage (H. Lowman (1997), Ann. Rev. Biophys. Biomol. Struct., 26, 401-424; and G. Smith et al., (1993), Meth. Enz., 217, 228-257). Randomised peptide libraries up to 38 amino acids in length have also been made, and longer peptides are achievable using this system. The peptide libraries that are produced using either of these strategies are then typically mixed with a pre-selected matrix-bound protein target. Peptides that bind are eluted, and their sequences are determined. From this information new peptides are synthesised and their biological properties can be assessed.

Other library expression systems that may be used include in vitro peptide generation libraries, such as: mRNA display (Roberts, & Szostak (1997), Proc. Natl. Acad. Sci. USA, 94, 12297-12302); ribosome display (Mattheakis et al., (1994), Proc. Natl. Acad. Sci. USA, 91, 9022-9026); and CIS display (Odegrip et al., (2004), Proc. Natl. Acad. Sci. USA, 101 2806-2810) amongst others.

The binding affinity of a selected modified igNAR peptide for a desired target ligand can be measured using any suitable technique known to the person of skill in the art, such as tryptophan fluorescence emission spectroscopy, isothermal calorimetry, surface plasmon resonance, or biolayer interferometry.

Beneficially, modified igNAR peptides of the invention have μM (e.g. less than 100 μM, less than 10 μM or about 1 μM) or tighter binding affinity for a target ligand, such as nM (e.g. 100 nM or lower, or 10 nM or lower) binding affinity.

Screening and Selection of Peptides from Libraries

In accordance with one aspect of the invention, igNAR nucleic acid libraries encoding a plurality of modified igNAR peptides particularly variable domains/fragments) are synthesised and initially selected for their ability to bind a desired target ligand. In a particularly advantageous method the peptides are displayed on the surface of phage particles by a phage display system, in which each modified igNAR peptide is expressed as a fusion protein to phage pIII coat protein.

The ligand may be a naturally or non-naturally occurring molecule, such as an organic small molecule, peptide or protein sequence. It may be a whole molecule or a part of a larger molecule (e.g. a domain, fragment or epitope of a protein), and may be an intracellular or an extracellular target molecule. In a beneficial embodiment the target ligand is an albumin protein or fragment thereof. The albumin is suitably a mammalian albumin, more suitably a primate albumin and most suitably a human albumin, such as HSA.

Conveniently, to aid in the separation of ligand-bound modified igNAR peptides from free peptides, the ligands may be associated with or otherwise attached to a solid support. By way of example, the solid support may be the surface of a plate, tube or well; alternatively the solid support may be a bead, such as a magnetic or agarose bead. In one example, the bead is a polystyrene-coated magnetic bead. The solid support may be coated with the ligand using any appropriate method. For instance, a ligand may be added to magnetic beads, for example, TALON® magnetic beads (Invitrogen, USA), in suitable buffer (such as PBS) and incubated for a period of time. The incubation can conveniently be carried out at room temperature whilst mixing on a rotary mixer. Before use the beads may be washed, for example, three times with PBS buffer.

The ligand (preferably immobilised) is then contacted with the library of modified igNAR peptides, typically by incubating the phage particles with expressed igNAR peptides on their surface with the ligand.

After a suitable incubation time, phage particles that are not associated with ligand are removed (e.g. by aspiration), typically, with one or more washing steps using suitable buffers and/or detergents; or by any other means known to the person of skill in the art. A convenient buffer is PBS, but other suitable buffers known in the art may also be used. By washing the mixture, library members that are incapable of associating with the target ligand (or which associate too weakly to remain associated under washing conditions) can be removed from the selection.

At least one round of expression, binding and selection is performed in order to enrich the population of modified igNAR peptides (and their associated phage particles) for the desired binding activity. Typically, 2, 3, 4, 5 or more rounds of selection may be carried out. In each (subsequent) round of selection certain criteria, particularly binding conditions, may be modified: for example, to enhance the selection of modified igNAR peptides having desirable properties, such as high affinity, increased specificity and so on.

At the end of each round of selection and at the end of the procedure, the ligand-associated modified igNAR peptides may then be recovered and individually characterised by sequencing the associated nucleic acid contained within the phage particle. Optionally, the peptides may be further characterised by expressing or synthesising the encoded igNAR peptide to confirm the desired ligand-binding properties. Advantageously, the modified igNAR peptides and/or nucleic acids of the invention may be isolated. However, a mixed population of modified igNAR peptides may also be obtained, e.g. where more than one igNAR peptide sequence is capable of associating under the chosen conditions with the target ligand. In this event, the invention also encompasses a mixed population of modified igNAR peptides that bind a target ligand.

### Nucleic Acids and Peptides

The modified igNAR peptides, antibodies or fragments according to the invention and, where appropriate, the modified igNAR peptide conjugates—e.g. where the igNAR peptide is associated with another moiety (such as a non-igNAR peptide moiety)—may be produced by recombinant DNA technology and standard protein expression and purification procedures. Thus, the invention further provides nucleic acid molecules that encode the modified igNAR peptides of the invention as well as their derivatives, and nucleic acid constructs, such as expression vectors, that comprise nucleic acids encoding peptides and derivatives according to the invention.

The term “vector” is used to denote a DNA molecule that is either linear or circular, into which another nucleic acid (typically DNA) sequence fragment of appropriate size can be integrated. Such DNA fragment(s) can include additional segments that provide for transcription of a gene encoded by the DNA sequence fragment. The additional segments can include and are not limited to: promoters, transcription terminators, enhancers, internal ribosome entry sites, untranslated regions, polyadenylation signals, selectable markers, origins of replication and such like. A variety of suitable promoters for prokaryotic (e.g. the β-lactamase and lactose promoter systems, alkaline phosphatase, the tryptophan (trp) promoter system, lac, tac, T3, T7 promoters for E. coli) and eukaryotic (e.g. simian virus 40 early or late promoter, Rous sarcoma virus long terminal repeat promoter, cytomegalovirus promoter, adenovirus late promoter, EG-1a promoter) hosts are available. Expression vectors are often derived from plasmids, cosmids, viral vectors and yeast artificial chromosomes; vectors are often recombinant molecules containing DNA sequences from several sources.

Specific embodiments of the present invention provide for an expression vector that encodes a modified igNAR peptide or fragment. Accordingly, the DNA encoding the relevant peptide of the invention can be inserted into a suitable expression vector (e.g. pGEM®, Promega Corp., USA), where it is operably linked to appropriate expression sequences, and transformed into a suitable host cell for protein expression according to conventional techniques (Sambrook J. et al., Molecular Cloning: a Laboratory Manual, Cold Spring Harbor Press, Cold Spring Harbor, N.Y.). Suitable host cells are those that can be grown in culture and are amenable to transformation with exogenous DNA, including bacteria, fungal cells and cells of higher eukaryotic origin, preferably mammalian cells. To aid in purifying the peptides of the invention, the igNAR peptide (and corresponding nucleic acid) of the invention may include a purification sequence, such as a His-tag. In addition, or alternatively, the modified igNAR peptides may, for example, be grown in fusion with another protein and purified as insoluble inclusion bodies from bacterial cells. This is particularly convenient when the modified igNAR peptide to be synthesised may be toxic to the host cell in which it is to be expressed. Alternatively, modified igNAR peptides may be synthesised in vitro using a suitable in vitro (transcription and) translation system (e.g. the E. coli S30 extract system: Promega corp., USA).

The term “operably linked”, when applied to DNA sequences, for example in an expression vector or construct indicates that the sequences are arranged so that they function cooperatively in order to achieve their intended purposes, i.e. a promoter sequence allows for initiation of transcription that proceeds through a linked coding sequence as far as the termination sequence.

In one embodiment of the present invention the vector is suitable as a polypeptide library display vector, enabling the polypeptide gene product of the modified igNAR-encoding gene to remain associated with the vector following transcription.

Having selected and isolated a desired modified igNAR peptide, an additional functional group such as a second therapeutic molecule may then be attached to the igNAR peptide by any suitable means. For example, a modified igNAR peptide may be conjugated to any suitable form of therapeutic molecule, such as an antibody, enzyme or small chemical compound. This can be particularly useful in applications where the modified igNAR peptide of the invention is capable of targeting or associating with a particular cell or organism that can be treated by the second therapeutic molecule. A preferred form of therapeutic molecule that may be attached or linked to a peptide or nucleic acid of the invention is a biological molecule, such as a polynucleic acid (e.g. siRNA) or a protein or polypeptide sequence (e.g. an antibody). Typically a chemical linker will be used to link a nucleic acid molecule to a peptide. Modified igNAR peptides may also be conjugated to a molecule that recruits immune cells of the host. Such conjugated igNAR peptides may be particularly useful for use as cancer therapeutics.

In a further alternative, the igNAR peptide, heavy chain antibody or fragment may be directly conjugated to another antibody molecule, an antibody fragment (e.g. Fab, F(ab)₂, scFv etc.) or other suitable targeting agent, so that the modified igNAR peptide and any additional conjugated moieties are targeted to the specific cell population required for the desired treatment or diagnosis, producing a bi-functional binder.

### Therapeutic Compositions

A modified igNAR peptide of the invention may be incorporated into a pharmaceutical composition for use in treating an animal; preferably a human. A therapeutic peptide of the invention (or derivative thereof) may be used to treat one or more diseases or infections, dependent on what ligand was used to select modified igNAR peptides from an igNAR peptide framework library. Alternatively, a nucleic acid encoding the therapeutic peptide may be inserted into an expression construct and incorporated into pharmaceutical formulations/medicaments for the same purpose.

The therapeutic peptides of the invention may be particularly suitable for the treatment of diseases, conditions and/or infections that can be targeted (and treated) extracellularly, for example, in the circulating blood or lymph of an animal; and also for in vitro and ex vivo applications. Therapeutic nucleic acids of the invention may be particularly suitable for the treatment of diseases, conditions and/or infections that are more preferably targeted (and treated) intracellularly, as well as in vitro and ex vivo applications. As used herein, the terms “therapeutic agent” and “active agent” encompass both peptides and the nucleic acids that encode a therapeutic modified igNAR peptide of the invention.

A preferred modified igNAR peptide is adapted to bind albumin protein sequences, and most suitably the HSA protein. It has been shown that peptides/proteins that bind albumin (e.g. HSA) exhibit longer half-lives in vivo compared with the free form of the same peptide. Thus, the albumin-binding modified igNAR peptides of the invention have extended half-lives in vivo (e.g. in the blood), when bound to HSA. Advantageously, however, the beneficial extended half-life of the modified igNAR peptide in vivo can be passed on to another (unstable) biological molecule by associating, coupling or fusing the biological molecule to the albumin-binding igNAR peptide. In this way, the albumin-binding igNAR peptides of the invention can be used to extend the half-lives of biological molecules in vivo, such as in the human body.

Therapeutic uses and applications for the modified igNAR peptides and nucleic acids of the invention therefore include any disease or condition that requires repetitive treatment regimes or the frequent administration of a biological therapeutic agent: particularly where large dosages of the therapeutic agent are typically used so as to maintain desirable blood plasma levels of the therapeutic molecule. Thus, therapeutic applications that may benefit from the albumin-binding igNAR peptides of the invention include: the treatment of various neoplastic and non-neoplastic diseases and disorders (e.g. cancers/neoplastic diseases and related conditions); neurodegenerative diseases or disorders (e.g. multiple sclerosis); and diabetes and diabetic-related conditions.

One or more additional pharmaceutically acceptable carrier (such as diluents, adjuvants, excipients or vehicles) may be combined with the therapeutic peptide of the invention in a pharmaceutical composition. Suitable pharmaceutical carriers are described in “Remington's Pharmaceutical Sciences” by E. W. Martin. Pharmaceutical formulations and compositions of the invention are formulated to conform to regulatory standards and can be administered orally, intravenously, topically, or via other standard routes. Administration can be systemic or local.

When administered to a subject, a therapeutic agent is suitably administered as a component of a composition that comprises a pharmaceutically acceptable vehicle. Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are preferably sterile. Water is a suitable vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Pharmaceutical compositions, if desired, can also contain minor amounts of wetting or emulsifying agents, or buffering agents.

The medicaments and pharmaceutical compositions of the invention can take the form of liquids, solutions, suspensions, lotions, gels, tablets, pills, pellets, powders, modified-release formulations (such as slow or sustained-release), suppositories, emulsions, aerosols, sprays, capsules (for example, capsules containing liquids or powders), liposomes, microparticles or any other suitable formulations known in the art. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

Orally administered compositions may contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. When the composition is in the form of a tablet or pill, the compositions may be coated to delay disintegration and absorption in the gastrointestinal tract, so as to provide a sustained release of active agent over an extended period of time. Selectively permeable membranes surrounding an osmotically active driving compound are also suitable for orally administered compositions. In these dosage forms, fluid from the environment surrounding the capsule is imbibed by the driving compound, which swells to displace the agent or agent composition through an aperture. These dosage forms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. For oral formulations, the location of release may be the stomach, the small intestine (the duodenum, the jejunem, or the ileum), or the large intestine. One skilled in the art is able to prepare formulations that will not dissolve in the stomach, yet will release the material in the duodenum or elsewhere in the intestine. Suitably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 would be essential. Examples of the inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac, which may be used as mixed films.

To aid dissolution of the therapeutic agent or nucleic acid (or derivative) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants, when used, could be present in the formulation of the peptide or nucleic acid or derivative either alone or as a mixture in different ratios.

Typically, compositions for intravenous administration comprise sterile isotonic aqueous buffer. Where necessary, the compositions may also include a solubilising agent.

Another suitable route of administration for the therapeutic compositions of the invention is via pulmonary or nasal delivery.

Additives may be included to enhance cellular uptake of the therapeutic peptide (or derivative) or nucleic acid of the invention, such as the fatty acids oleic acid, linoleic acid and linolenic acid.

The therapeutic peptides or nucleic acids of the invention may also be formulated into compositions for topical application to the skin of a subject.

Modified igNAR peptides and nucleic acids of the invention may also be useful in non-pharmaceutical applications, such as in diagnostic tests, imaging, as affinity reagents for purification and as delivery vehicles.

The invention will now be further illustrated by way of the following non-limiting examples.

### EXAMPLES

Unless otherwise indicated, commercially available reagents and standard techniques in molecular biological and biochemistry were used.

### Materials and Methods

The following procedures used by the Applicant are described in Sambrook, J. et al., 1989, supra.: analysis of restriction enzyme digestion products on agarose gels and preparation of phosphate buffered saline. General purpose reagents were purchased from Sigma-Aldrich Ltd (Poole, Dorset, UK). Oligonucleotides were obtained from Sigma Genosys Ltd (Haverhill, Suffolk, UK) or Genelink Inc., (Hawthorne, N.Y., USA). Enzymes and polymerases were obtained from New England Biolabs (NEB, Cambridgeshire, UK). Chemicals and solvents were purchased from Fisher Scientific (Loughborough, Leicestershire, UK).

### Example 1

### A. Library Construction

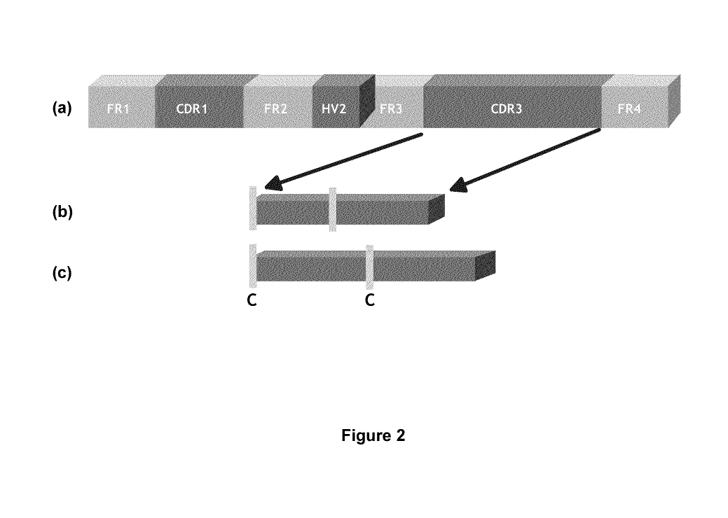

Four primary libraries based on wild-type Wobbegong igNAR protein variable domain fragment (FIGS. 1 and 2) having mutant CDR3 loop regions in the CDR3 region (i.e. between Tyr85 and Lys97) of the wild-type sequence were made. In this example, only the CDR3 region (SEQ ID NO: 87) was varied. This conservative approach was designed to reduce the chance of introducing hydrophobic patches (typical of “sticky” or non-specific clones), and to allow subsequent step-wise maturation of lead molecules.

To create the libraries, oligonucleotides were designed to encode CDR3 loops of 11, 13, 16 and 18 residues (SEQ ID NOs: 1 to 4, respectively). CDR3 peptide libraries of 11, 13 and 18 amino acids included a fixed cysteine residue of wild-type igNAR; while the 16 amino acid CDR3 library did not have a fixed cysteine residue (FIG. 1). All randomised amino acid positions between Tyr and Lys indicated in FIG. 1 were encoded in the library by an NNK codon (where N represents an equal mix of G, A, T and C; and K represents an equal mix of G and T) in the nucleic acid sequence.

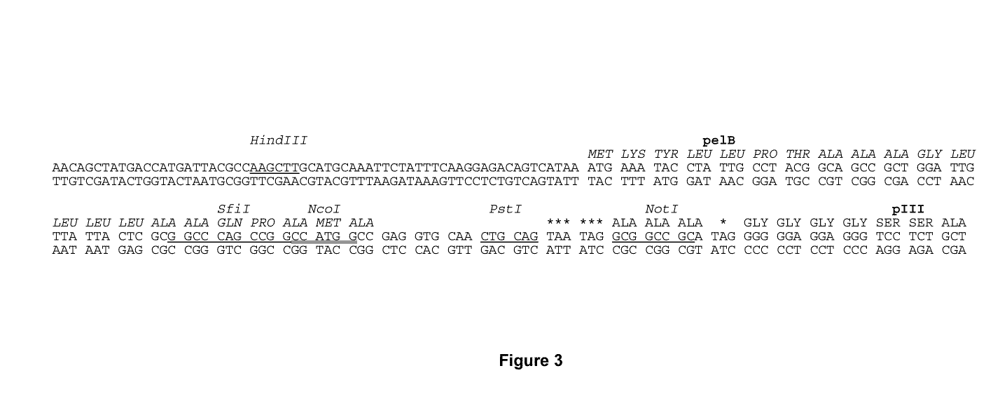

Libraries were PCR-generated using a GeneArt synthesised Wobbegong igNAR variable domain scaffold (amino acid and DNA sequences shown in FIG. 1), and cloned as NcoI-NotI digested fragments into similarly digested pSP1 phagemid pIII fusion vector derived from the pHEN1 pIII vector (Hoogenboom et al., 1991, Nucleic Acids Res., 19: 4133-4137). The pSP1 multiple cloning site is shown in FIG. 3.

(i) PCR Amplification of igNAR CDR3 Libraries

For the primary PCR amplifications 10×50 μl amplifications were set up for each CDR3 library using the appropriate REV oligonucleotide primer (SEQ ID NO: 1 to 4, as appropriate) and the GeneArtFOR primer (SEQ ID NO: 5). Each 50 μl reaction mixture contained 10 ng Wobbegong igNARGeneArt cDNA, 25 pmol of the appropriate forward and reverse primers, 0.1 mM dNTPs, 2.5 units Taq DNA polymerase, and 1×NEB PCR reaction buffer (20 mM Tris-HCl pH 8.8, 10 mM (NH₄)₂SO₄,10 mM KCl, 2 mM MgSO₄, 0.1% Triton X-100; NEB Ltd, Cambridge, UK). Reactions were performed for 30 PCR cycles of 94° C., 20 s; 60° C., 40 s; 72° C., 30 s, followed by 5 minutes at 72° C. Reaction products were purified using two Wizard PCR clean-up columns per repertoire (Promega Ltd, Southampton, UK), and eluted into 50 μl water per column.

### (ii) Pull-Through Re-Amplification of Selected DNA

To prepare the final igNAR DNA products, 40×50 μl amplifications were set up for each CDR3 loop library, using CDR3PTFOR (SEQ ID NO: 6) primer and CDR3PTREV primer (SEQ ID NO: 7). Each 50 μl reaction mixture contained approximately 25 ng primary CDR3 library Wobbegong igNAR DNA, 25 pmol of the appropriate forward and reverse primers, 0.1 mM dNTPs, 2.5 units Taq DNA polymerase, and 1×NEB PCR reaction buffer (20 mM Tris-HCl pH 8.8, 10 mM (NH₄)₂SO₄, 10 mM KCl, 2 mM MgSO₄, 0.1% Triton X-100; NEB Ltd, Cambridge, UK). Reactions were performed for 25 cycles of 94° C., 20 s; 60° C., 40 s; 72° C., 30 s, followed by 5 minutes at 72° C. Reaction products were purified using four Wizard PCR clean-up columns per library (Promega Ltd, Southampton, UK), and eluted into 100 μl water per column.

(iii) Cloning into Vector pSP1

Each of the four libraries, and 250 μg pSP1 vector DNA were digested with enzymes NcoI and NotI (100 units each enzyme) for 5 hours at 37° C. (NEB, Cambridge, UK), and purified using one Wizard PCR clean-up column per library, and four Wizard PCR clean-up columns for the digested vector DNA (Promega Ltd, Southampton, UK). Each DNA sample was then eluted into 100 μl water. Half of each digested library DNA was ligated overnight at 16° C. in 400 μl with 50 μg of NcoI-NotI cut pSP1 vector and 4000 U of T4 DNA ligase (NEB Ltd, Southampton, UK). After incubation the ligations were adjusted to 200 μl with nuclease free water, and DNA precipitated with 1 μl 20 mg/ml glycogen, 100 μl 7.5M ammonium acetate and 900 μl ice-cold (−20° C.) absolute ethanol, vortex mixed and spun at 13,000 rpm for 20 minutes in a microfuge to pellet DNA. The pellets were washed with 500 μl ice-cold 70% ethanol by centrifugation at 13,000 rpm for 2 minutes, then vacuum dried and re-suspended in 100 μl DEPC-treated water. 1 μl aliquots of each library were electroporated into 80 μl E. coli (TG1). Bacterial cells were grown in 1 ml SOC medium per cuvette for 1 hour at 37° C., and plated onto 2×TY agar plates supplemented with 2% glucose and 100 μg/ml ampicillin. 10⁻⁴, 10⁻⁵ and 10⁻⁶ dilutions of the electroporated bacteria were also plated to assess library size. Colonies were allowed to grow overnight at 30° C. Combined library size was of the order of 2×10¹⁰ clones with >95% with in-frame inserts.

The resultant naïve igNAR variable domain peptide libraries have the sequences of SEQ ID NOs: 89 to 92, respectively, for the 11, 13, 16 and 18 residue CDR3 loop peptide variants.

TABLE 1
Sequences of CDR3 loop library and PCR primers: fixed cysteine
in CDR3 loop region shown in bold. Peptide library sequences for
libraries having CDR3 loop lengths of 11, 13, 16 and 18 amino
acids (SEQ ID NOs: 89 to 92, respectively), X represents any
amino acid in the library, full CDR3 loops shown in bold.

| SEQ | |
|---|---|
| ID NO: | Sequence |
| 1 | 11-CDR3 oligonucleotide |
| | TACGGTGCCAGCTCCCTCMNNMNNMNNMNNMNNMNNMNNGCAMNNM |
| | NNMNNTGCTTTACACTTATACGTGCC |
| 2 | 13-CDR3 oligonucleotide |
| | TACGGTGCCAGCTCCCTCMNNMNNMNNMNNMNNMNNMNNMNNMNNG |
| | CAMNNMNNMNNTGCTTTACACTTATACGTGCC |
| 3 | 16-CDR3 oligonucleotide |
| | TACGGTGCCAGCTCCCTCMNNMNNMNNMNNMNNMNNMNNMNNMNNM |
| | NNMNNMNNMNNMNNMNNMNNTGCTTTACACTTATACGTGCC |
| 4 | 18-CDR3 oligonucleotide |
| | TACGGTGCCAGCTCCCTCMNNMNNMNNMNNMNNMNNMNNMNNMNNM |
| | NNMNNMNNMNNGCAMNNMNNMNNMNNTGCTTTACACTTATACGTGCC |
| 5 | GeneArtFOR |
| | GGCCGTCAAGGCCACGTGTCTTGTCC |
| 6 | CDR3PTFOR |
| | AAAAAAGCCATGGCAAGGGTGGACCAAACACCAAGAATAGCAACAAAA |
| | GAGACGGGCGAATCACTGACCATCAATTGCGTCCTAAG |
| 7 | CDR3PTREV |
| | TAGGCCAATTGCGGCCGCACCTCCTTTCACGGTTAATACGGTGCCAGCT |
| | CCCTC |
| 89 | RVDQTPRIATKETGESLTINCVLRDTACALDSTNWYRTKLGSTKEQTISIGG |
| | RYSETVDEGSNSASLTIRDLRVEDSGTYKCKAXXXCXXXXXXXEGAGTVLT |
| | VK |
| 90 | RVDQTPRIATKETGESLTINCVLRDTACALDSTNWYRTKLGSTKEQTISIGG |
| | RYSETVDEGSNSASLTIRDLRVEDSGTYKCKAXXXCXXXXXXXXXEGAGTV |
| | LTVK |
| 91 | RVDQTPRIATKETGESLTINCVLRDTACALDSTNWYRTKLGSTKEQTISIGG |
| | RYSETVDEGSNSASLTIRDLRVEDSGTYKCKAXXXXXXXXXXXXXXXXEGA |
| | GTVLTVK |
| 92 | RVDQTPRIATKETGESLTINCVLRDTACALDSTNWYRTKLGSTKEQTISIGG |
| | RYSETVDEGSNSASLTIRDLRVEDSGTYKCKAXXXXCXXXXXXXXXXXXXE |
| | GAGTVLTVK |

### (iv) Phage Amplification

Separate phage stocks were prepared for each CDR3 library. The bacteria were then scraped off the plates into 50 ml 2×TY broth supplemented with 20% glycerol, 2% glucose and 100 μg/ml ampicillin. 1 ml of bacterial medium was added to a 50 ml 2×TY culture broth supplemented with 1% glucose and 100 μg/ml ampicillin and infected with 10¹¹ kanamycin resistance units (kru) M13K07 helper phage at 37° C. for 30 minutes without shaking, then for 30 minutes with shaking at 200 rpm. Infected bacteria were transferred to 200 ml 2×TY broth supplemented with 25 μg/ml kanamycin, 100 μg/ml ampicillin, and 20 μM IPTG, then incubated overnight at 30° C., shaking at 200 rpm. Bacteria were pelleted at 4000 rpm for 20 minutes in 50 ml Falcon tubes, and 40 ml 2.5M NaCl/20% PEG 6000 was added to 400 ml of particle supernatant, mixed vigorously and incubated on ice for 1 hour to precipitate phage particles. Particles were pelleted at 11000 rpm for 30 minutes in 250 ml Oakridge tubes at 4° C. in a Sorvall RC5B centrifuge, then resuspended in 40 ml water and 8 ml 2.5M NaCl/20% PEG 6000 added to reprecipitate particles, then incubated on ice for 20 minutes. Particles were again pelleted at 11000 rpm for 30 minutes in 50 ml Oakridge tubes at 4° C. in a Sorvall RC5B centrifuge, then resuspended in 5 ml PBS buffer, after removing all traces of PEG/NaCl with a pipette. Bacterial debris was removed by a 5 minute 13500 rpm spin in a microcentrifuge. The supernatant was filtered through a 0.45 μm polysulfone syringe filter, adjusted to 20% glycerol and stored at −70° C.

### B. Selection Against Human Serum Albumin

Free igNAR has an in vivo half-life in humans of around 10 minutes, with almost total clearance via the kidney by 30 minutes. Albumin binding igNARs may have value in extending the in vivo half-life of therapeutic peptides and proteins lacking PEGylation or antibody Fc regions. Selections were, therefore, carried out using the four libraries described in Example 1, in order to select non-natural igNAR proteins having mutated CDR3 loop regions and capable of binding to human serum albumin (HSA).

### (i) Library Selections

NUNC Star immunotubes were coated overnight with HSA (SIGMA-Aldrich) at 100 μg/ml PBS (2 ml/tube) at 4° C., and then rinsed three times in PBS (by filling and emptying tubes). Tubes were blocked at room temperature for 1 hour with 2% milk powder/PBS, then rinsed three times in PBS. An aliquot of approximately 10¹³ a.r.u. pooled igNAR library stock was adjusted to 2 ml with 2% milk powder/PBS and added to the coated, blocked tube for two hours on a blood mixer. The tube was then washed ten times in PBS/0.1% Tween 20, then ten times in PBS. After the final wash, bound phage were eluted with 1 ml of freshly prepared 0.1M triethylamine for 10 minutes, the beads were captured, and eluted particles transferred to 0.5 ml 1M Tris-HCl pH 7.4. Neutralised particles were added to 10 ml log phase TG1 E. coli bacteria and incubated at 37° C. without shaking for 30 minutes, then with shaking at 200 rpm for 30 minutes. 10⁻³, 10⁻⁴ and 10⁻⁵ dilutions of the infected culture were prepared to estimate the number of particles recovered; the remainder was then spun at 4000 rpm for 10 minutes, and the resultant pellet resuspended in 300 μl 2×TY medium by vortex mixing. Bacteria were plated onto 2×TY agar plates supplemented with 2% glucose and 100 μg/ml ampicillin, and colonies allowed to grow overnight at 30° C.

Finally, a 100-fold concentrated phage stock was prepared from a 100 ml amplified culture of these bacteria as described above, and 0.5 ml used in two further rounds of selection prior to screening of the third round output.

### (ii) ELISA Identification of Binding Clones

Binding clones were identified by ELISA of 96 individual phage cultures prepared by picking individual TG1 bacteria clones from the third round of selection into 100 μl/well of 2×TY culture broth supplemented with 1% glucose and 100 μg/ml ampicillin in a 96 well plate. Plates are placed into an orbital shaker and incubated overnight at 37° C./200 rpm. 25 μl of culture medium from each well was transferred into a 96-well deep well plate containing 300 μl growth medium and grown for 5 to 6 hours, and then 25 μl/well 2×TY culture broth supplemented with 1% glucose and 100 μg/ml ampicillin and approximately 10⁸ M13K07 kru was added to each well. Infection was carried out at 37° C. without shaking for 30 minutes, then with shaking for a further 30 minutes. Plates were spun at 2300 rpm in a microplate centrifuge to pellet infected bacteria, and the cultures were then induced by adding 300 μl/well 2×TY broth supplemented with 25 μg/ml kanamycin, 100 μg/ml ampicillin and 100 μM IPTG, and then incubated at 30° C./200 rpm overnight.

A Dynatech Immulon 4 ELISA plate was coated with 500 ng/well HSA in 100 μl/well PBS overnight at 4° C. The plate was washed twice with 200 μl/well PBS and blocked for 1 hour at 37° C. with 200 μl/well 2% milk powder/PBS and then washed twice with 200 μl/well PBS. 50 μl phage culture supernatant was added to each well containing 50 μl/well 4% Marvel/PBS, and allowed to bind for 1 hour at room temperature. The plate was washed two times with 200 μl/well PBS/0.1% Tween 20, and then two times with 200 μl/well PBS. Bound phage were detected with 100 μl/well, 1:5000 diluted anti-M13-HRP conjugate (Pharmacia) in 2% Marvel/PBS for 1 hour at room temperature and the plate washed as above. The plate was developed for 5 minutes at room temperature with 100 μl/well freshly prepared TMB (3,3′,5,5′-Tetramethylbenzidine) substrate buffer (0.005% H₂O₂, 0.1 mg/ml TMB in 24 mM citric acid/52 mM sodium phosphate buffer pH 5.2). The reaction was stopped with 100 μl/well 12.5% H₂SO₄ and read at 450 nm.

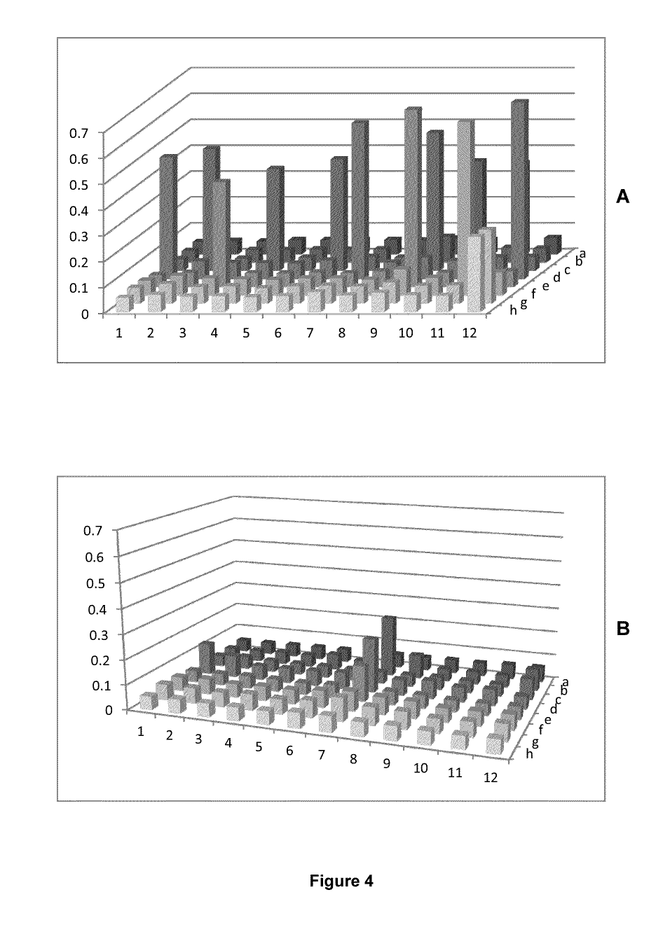

Out of 96 clones tested, at least 12 wells gave signals greater than twice background. FIG. 4A shows the binding strength of selected clones for HSA; and FIG. 4B shows the background binding strength of the same clones for blocked wells that do not contain target protein (average background=0.05).

(iii) Albumin Binding Clone Sequences

Twelve clones identified in Example 1B(ii) above were sequenced to determine the novel CDR3 sequences that bound to HSA. All twelve clones contained the same amino acid sequence in the CDR3 loop region (SEQ ID NO: 9). A representative full igNAR clone has the sequence of SEQ ID NO: 8. Another representative clone (clone B10: see also FIG. 4A) has the amino acid sequence of SEQ ID NO: 10).

TABLE 2
Peptide sequence of albumin binding mutant igNAR protein: mutant CDR3 loop
(SEQ ID NO: 9) shown underlined in SEQ ID NOs: 8 and 10. Amino acid sequence
differences between SEQ ID NOs: 8 and 10 (out side of CDR 3 and CDR1 loops)
identified in bold in SEQ ID NO: 10.

| SEQ | |
|---|---|
| ID NO: | Sequence |
| 8 | RVDQTPRIATKETGESLTINCVLRDTACALDSTNVVYRTKLGSTKEQTISIGGRYSETV |
| | DEGSNSASLTIRDLRVEDSGTYKCKAAITPFDNWYECLGTRAEGAGTVLTVK |
| 9 | AITPFDNWYECLGTRA |
| 10 | RVDQTPRIATKETGESLTINCVLRDTACALDSTNWYRTKLGSTKEQTISIGGRYSETA |
| | DEGSNPASLTIRDLRVEDSGTYKCKAAITPFDNWYECLGTRAEGAGTVLTVK |

### C. Albumin-Binding Peptide Specificity

To assess the specificity of the selected HSA binding peptides for albumin rather than non-target proteins, further ELISA assays were carried out similar to those already described above.

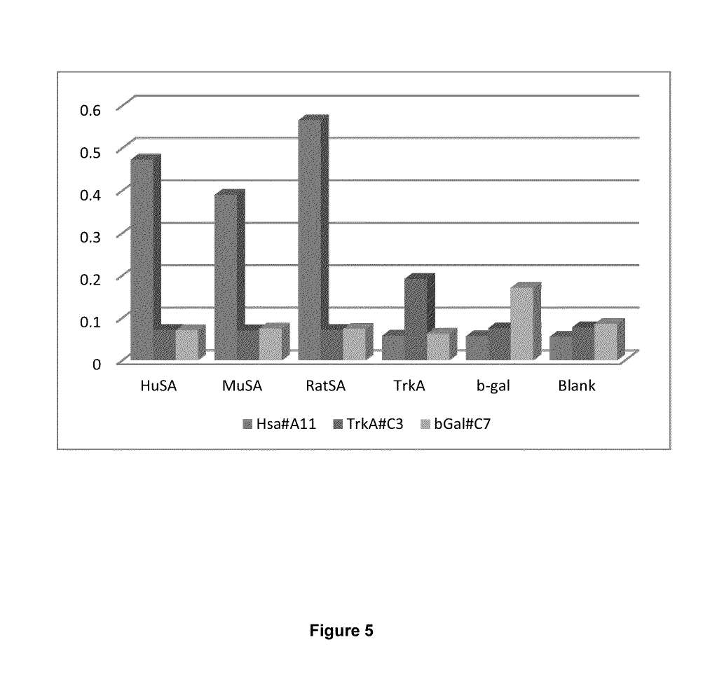

A representative phage clone, #A11, was grown up in a 10 ml culture volume as described above, and phage-igNAR specificity examined by ELISA against human, murine and rat serum albumin (coated at 50 μg/ml), plus blocked plastic, β-galactosidase and trkA-Fc fusion protein (coated at 2 μg/ml). The results confirm the specificity of this clone for albumin binding over non-target proteins (see FIG. 5).

### Example 2

### Second Generation CDR3 Loop Libraries

Second generation CDR3 loop libraries were constructed similarly to those described in Example 1, except randomised amino acid positions were encoded using trinucleotide-containing oligonucleotides.

The second generation libraries were screened for HSA binding in an analogous manner to that described in Example 1.

### Example 3

### Third Generation Libraries

### Maturation Via CDR1 Loop Randomisations

Third generation igNAR variable domain mutant protein libraries having randomised CDR1 loop regions can be constructed and screened for binding to albumin in order to fine tune the binding affinity and specificity of the mutant proteins selected in Examples 1 and 2. Accordingly, for one of the third generation libraries the HSA-binding protein sequence of SEQ ID NO: 8 is taken as the base template/framework. In another third generation library the modified igNAR peptide clone B10 (see FIG. 4A; SEQ ID NO: 10) was used as the scaffold for CDR1 loop library selection.

CDR1 loop libraries were constructed by randomising the peptide sequence of the CDR1 loop (SEQ ID NO: 11) in one or more (up to all 6) of positions 19, 20 and 22 to 25 of the Wobbegong sequence shown in FIG. 1 (see also Table 3). The cysteine residue at position 21 was invariant.

The third generation libraries were otherwise constructed and screened for binding to HSA in an analogous manner to that described in Example 1, as described below.

### A. Library Construction

An initial HSA-binding clone (B10; SEQ ID NO: 10; Table 2) from selection of Example 2 was used as a template from which to generate matured clones via replacement of the CDR1 loop, i.e. between Ile19 and Asp25.

To create the library a degenerate oligonucleotide was designed to encode 6 randomised residues at positions 19, 20, 22, 23, 24 and 25 with a fixed cysteine at position 21 in the igNAR sequence (SEQ ID NO: 12). The NNK codon was used for diversifying the selected library positions such that all possible naturally-occurring amino acids were encoded between Iso19 and Asp25 in the library (excluding the fixed cysteine).

Libraries were generated via PCR using B10 template DNA and cloned as NcoI-NotI digested fragments into similarly digested pSP1 phagemid pIII fusion vector.

(i) PCR Amplification of igNAR CDR1 Libraries

For the primary PCR amplifications 10×50 μl amplifications were set up for each CDR1 library using the appropriate forward and reverse oligonucleotide primers (SEQ ID NOs: 12 and 13, respectively). Each 50 μl reaction mixture contained 10 ng clone B10 DNA, 25 pmol of the appropriate forward and reverse primers, 0.1 mM dNTPs, 2.5 units Taq DNA polymerase, and 1×NEB PCR reaction buffer (20 mM Tris-HCl pH 8.8, 10 mM (NH₄)₂SO₄,10 mM KCl, 2 mM MgSO₄, 0.1% Triton X-100: NEB Ltd, Cambridge, UK). Reactions were performed for 30 PCR cycles of 94° C., 20 s; 60° C., 40 s; 72° C., 30 s, followed by 5 minutes at 72° C. Reaction products were purified using two Wizard PCR clean-up columns per library (Promega Ltd, Southampton, UK), and eluted into 50 μl water per column.

### (ii) Pull-Through Re-Amplification of Selected DNA

To prepare the final igNAR DNA products, 10×50 μl amplifications were set up using the appropriate forward and reverse oligonucleotide primers (SEQ IDs NO: 14 and 15). Each 50 μl reaction mixture contained approximately 25 ng primary CDR1 library igNAR DNA, 25 pmol of the appropriate forward and reverse primers, 0.1 mM dNTPs, 2.5 units Taq DNA polymerase, and 1×NEB PCR reaction buffer (20 mM Tris-HCl pH 8.8, 10 mM (NH₄)₂SO₄,10 mM KCl, 2 mM MgSO₄, 0.1% Triton X-100: NEB Ltd, Cambridge, UK). Reactions were performed for 25 cycles of 94° C., 20 s; 60° C., 40 s; 72° C., 30 s, followed by 5 minutes at 72° C. Reaction products were purified using four Wizard PCR clean-up columns per library (Promega Ltd, Southampton, UK), and eluted into 100 μl water per column.

TABLE 3
Peptide sequence of CDR1 loop of igNAR protein (SEQ ID NO: 11) shown
underlined in modified igNAR peptide sequence of SEQ ID NO: 10. Primer/
oligonucleotide sequences used in construction of CDR1 loop libraries.

| SEQ | |
|---|---|
| ID NO: | Sequence |
| 10 | RVDQTPRIATKETGESLTINCVLRDTACALDSTNWYRTKLGSTKEQTISIGG |
| | RYSETADEGSNPASLTIRDLRVEDSGTYKCKAAITPFDNWYECLGTRAEGA |
| | GTVLTVK |
| 11 | INCVLRD |
| 12 | GACCATCAATTGCGTCCTAAGAGATNNKNNKTGTNNKNNKNNKNNKACG |
| | AATTGGTATCGGACAAAATTGGG |
| 13 | CAACTTTCAACAGTTTCAGCAGAGG |
| 14 | AGAATTTCCATGGCACTCGTGGACCAAACACCAAGAATAGCAACAAAAG |
| | AGACGGGCGAATCACTGACCATCAATTGCGTCCTAAG |
| 15 | TAGGCCAATTGCGGCCGCACCTCCTTTCACGGTTAATACGGTGCCAGCT |
| | CCCTC |

(iii) Cloning into Vector pSP1

Pull-through PCR products and 40 μg pSP1 vector DNA were digested with enzymes NcoI and NotI (20 units each enzyme) for 5 hours at 37° C. (NEB, Cambridge, UK), and purified using one Wizard PCR clean-up column per library, and four Wizard PCR clean-up columns for the digested vector DNA (Promega Ltd, Southampton, UK). DNA sample was then eluted into 100 μl water. Digested library DNA was ligated overnight at 16° C. in 200 μl with 30 μg of NcoI-NotI cut pSP1 vector and 500 U of T4 DNA ligase (NEB Ltd, Southampton, UK). After incubation the DNA was precipitated with 1 μl 20 mg/ml glycogen, 100 μl 7.5M ammonium acetate and 900 μl ice-cold (−20° C.) absolute ethanol, vortex mixed and spun at 13,000 rpm for 20 minutes in a microfuge to pellet DNA. The pellets were washed with 500 μl ice-cold 70% ethanol by centrifugation at 13,000 rpm for 2 minutes, then vacuum dried and re-suspended in 100 μl DEPC-treated water. 1 μl aliquots of each library were electroporated into 25 μl E. coli (TG1). Bacterial cells were grown in 1 ml SOC medium per cuvette for 1 hour at 37° C., and plated onto 2×TY agar plates supplemented with 2% glucose and 100 μg/ml ampicillin. 10⁻⁴, 10⁻⁵ and 10⁻⁶ dilutions of the electroporated bacteria were also plated to assess library size. Colonies were allowed to grow overnight at 30° C. Library size was of the order of 2×10⁹ clones with >95% with in-frame inserts.

### (iv) Phage Amplification

Bacteria were scraped off the plates into 50 ml 2×TY broth supplemented with 20% glycerol, 2% glucose and 100 μg/ml ampicillin. 1 ml of bacterial medium was added to a 50 ml 2×TY culture broth supplemented with 1% glucose and 100 μg/ml ampicillin and infected with 10¹¹ kanamycin resistance units (kru) M13K07 helper phage at 37° C. for 30 minutes without shaking, then for 30 minutes with shaking at 200 rpm. Infected bacteria were transferred to 200 ml 2×TY broth supplemented with 25 μg/ml kanamycin, 100 μg/ml ampicillin, and 20 μM IPTG, then incubated overnight at 30° C., shaking at 200 rpm. Bacteria were pelleted at 4000 rpm for 20 minutes in 50 ml Falcon tubes, and 40 ml 2.5M NaCl/20% PEG 6000 was added to 400 ml of particle supernatant, mixed vigorously and incubated on ice for 1 hour to precipitate phage particles. Particles were pelleted at 11000 rpm for 30 minutes in 250 ml Oakridge tubes at 4° C. in a Sorvall RC5B centrifuge, then re-suspended in 40 ml water and 8 ml 2.5M NaCl/20% PEG 6000 added to re-precipitate particles, then incubated on ice for 20 minutes. Particles were again pelleted at 11000 rpm for 30 minutes in 50 ml Oakridge tubes at 4° C. in a Sorvall RC5B centrifuge, then re-suspended in 5 ml PBS buffer, after removing all traces of PEG/NaCl with a pipette. Bacterial debris was removed by a 5 minute 13500 rpm spin in a microcentrifuge. The supernatant was filtered through a 0.45 μm polysulfone syringe filter, adjusted to 20% glycerol and stored at −70° C.

### B. Selection Against Human Serum Albumin

As already noted, albumin binding igNARs may have value in extending the in vivo half-life of therapeutic peptides and proteins lacking PEGylation or antibody Fc regions. Libraries of igNAR peptides randomised in the CDR3 loop region have already been screened for their ability to bind to HSA. By further diversifying the selected CDR3-modified igNAR peptides in the CDR1 loop region and selecting for binding to HAS it may be possible to identify improved, matured modified igNAR peptides for HSA-binding.

### (i) Maturation Library Selections

NUNC Star immunotubes were coated overnight with HSA (SIGMA-Aldrich) at 0.6 μg/ml PBS (2 ml/tube) at 4° C., and then rinsed three times in PBS (by filling and emptying tubes). Tubes were blocked at room temperature for 1 hour with 2% milk powder/PBS, then rinsed three times in PBS. An aliquot of approximately 10¹³ a.r.u. library stock was adjusted to 1 ml with 2% milk powder/PBS and added to the coated, blocked tube for two hours on a blood mixer. The tube was then washed ten times in PBS/0.1% Tween 20, then ten times in PBS. After the final wash, bound phage were eluted in a step-wise fashion by specific elution. First, tubes were incubated for 1 hour at room temperature with HSA at a concentration of 1.2 mg/ml in PBS. Supernatant was removed and added to 10 ml log phase TG1 E. coli bacteria and incubated at 37° C. without shaking for 30 minutes, then with shaking at 200 rpm for 30 minutes. Second, tubes were incubated for 1 hour at room temperature with HSA at a concentration of 12 mg/ml in PBS. Supernatant was removed and added to 10 ml log phase TG1 E. coli bacteria and incubated at 37° C. without shaking for 30 minutes, then with shaking at 200 rpm for 30 minutes. Finally remaining phage were eluted with 1 ml of freshly prepared 0.1M triethylamine for 10 minutes, eluted particles were then transferred to 0.5 ml 1M Tris-HCl pH 7.4. Neutralised particles were added to 10 ml log phase TG1 E. coli bacteria and incubated at 37° C. without shaking for 30 minutes, then with shaking at 200 rpm for 30 minutes. 10⁻³, 10⁴ and 10⁻⁵ dilutions of the infected cultures were prepared to estimate the number of particles recovered; the remainder was then spun at 4000 rpm for 10 minutes, and the resultant pellet re-suspended in 300 μl 2×TY medium by vortex mixing. Bacteria were plated onto 2×TY agar plates supplemented with 2% glucose and 100 μg/ml ampicillin, and colonies allowed to grow overnight at 30° C.

Finally, a 100-fold concentrated phage stock was prepared from a 100 ml amplified culture of selected bacteria as described above, and 0.5 ml used in one further round of selection.

The second round of screening was performed as described above except that HSA was coated on NUNC Star immunotubes at 6 ng/ml.

### C. ELISA Identification of Binding Clones

Binding clones were identified by ELISA of 96 individual phage cultures prepared by picking individual TG1 bacteria clones from the third round of selection into 100 μl/well of 2×TY culture broth supplemented with 1% glucose and 100 μg/ml ampicillin in a 96 well plate. Plates are placed into an orbital shaker and incubated overnight at 37° C./200 rpm. 25 μl of culture medium from each well was transferred into a 96-well deep well plate containing 300 μl growth medium and grown for 5 to 6 hours, and then 25 μl/well 2×TY culture broth supplemented with 1% glucose and 100 μg/ml ampicillin and approximately 10⁸ M13K07 kru was added to each well. Infection was carried out at 37° C. without shaking for 30 minutes, then with shaking for a further 30 minutes. Plates were spun at 2300 rpm in a microplate centrifuge to pellet infected bacteria, and the cultures were then induced by adding 300 μl/well 2×TY broth supplemented with 25 μg/ml kanamycin, 100 μg/ml ampicillin and 100 μM IPTG, and then incubated at 30° C./200 rpm overnight.

A Dynatech Immulon 4 ELISA plate was coated with 50 ng/well HSA in 100 μl/well PBS overnight at 4° C. The plate was washed twice with 200 μl/well PBS and blocked for 1 hour at 37° C. with 200 μl/well 2% milk powder/PBS and then washed twice with 200 μl/well PBS. 50 μl phage culture supernatant was added to each well containing 50 μl/well 4% Marvel/PBS, and allowed to bind for 1 hour at room temperature. The plate was washed two times with 200 μl/well PBS/0.1% Tween 20, and then two times with 200 μl/well PBS. Bound phage were detected with 100 μl/well, 1:5000 diluted anti-M13-HRP conjugate (Pharmacia) in 2% Marvel/PBS for 1 hour at room temperature and the plate washed as above. The plate was developed for 5 minutes at room temperature with 100 μl/well freshly prepared TMB substrate buffer (0.005% H₂O₂, 0.1 mg/ml TMB in 24 mM citric acid/52 mM sodium phosphate buffer pH 5.2). The reaction was stopped with 100 μl/well 12.5% H₂SO₄ and read at 450 nm.

### (ii) Albumin Binding Clone Sequences

Thirty four clones were identified as binding to HSA and were sequenced to determine the novel CDR1 sequences within the original HSA-binding sequence of SEQ ID NO: 10, and the CDR1 loop sequences identified (SEQ ID NOs: 16 to 50) are shown in Table 4. All clones contained the same amino acid sequence in the CDR3 loop region (SEQ ID NO: 9) and, except at the CDR1 region, were otherwise the same sequence of SEQ ID NO: 10. The full-length selected third generation igNAR peptides have the sequences of SEQ ID NOs: 51 to 85.

```
TABLE 4
Peptide sequences of albumin binding mutant
igNAR variable domain peptide CDR1
    loop regions (SEQ ID NOs: 16 to 50).
  Full length third generation igNAR
variable domain peptide sequences
    obtained by replacing wild-type
CDR1 region (i.e. SEQ ID 5 NO: 11) within
SEQ ID NO: 10 with each of the respective
CDR1 loop regions (SEQ ID NOs: 51 to 85).
```

| SEQ | | SEQ | |
|---|---|---|---|
| ID NO: | Sequence | ID NO: | Sequence |
| 11 | INCVLRD | 33 | SMCHLQE |
| 16 | TLCHMSF | 34 | TMCHWQD |
| 17 | TICQIST | 35 | TLCHIAV |
| 18 | SLCQMHT | 36 | TLCHMAW |
| 19 | TICSLAF | 37 | TLCHLYS |
| 20 | SLCWMYI | 38 | TLCHPAW |
| 21 | TICWQTE | 39 | TLCNIEL |
| 22 | SLCWMMD | 40 | SLCGIHE |
| 23 | TLCTMIW | 41 | TFCILHD |
| 24 | SMCHMTQ | 42 | TACALDS |
| 25 | SLCGIHE | 43 | SMCWAII |
| 26 | TICLQEE | 44 | TLCVVPQ |
| 27 | TLCGAAD | 45 | TMCLFMV |
| 28 | TLCRMTG | 46 | TLCDLMI |
| 29 | SLCHIKD | 47 | TICLQEE |
| 30 | SMCHMTQ | 48 | TLCGAAD |
| 31 | TMCEFQD | 49 | SLCHISF |
| 32 | TFCELAE | 50 | TLCIMTS |

### Example 4

The effect of albumin-binding activity on igNAR protein half-life in vivo is tested by mixing the mutant igNAR proteins of the invention, e.g. the peptides of SEQ ID NO: 8 and 10 and the peptides sequenced in Example 3 above, with albumin to prepare albumin-mutant igNAR complexes. The half-life of the albumin-mutant igNAR complex in vivo can then be measured and compared to that of the free mutant igNAR proteins in a mammal.

Binding to albumin is found to extend the in vivo half-life of the mutant igNAR protein compared to that of free protein.

### Example 5

The effect of albumin-binding activity on the half-life of a biological molecule (or “biological”) in vivo is tested by conjugating the mutant igNAR proteins of the invention, such as the selected peptide of SEQ ID NO: 8 and 10 and the peptides sequenced in Example 3 above, to the biological molecule. Any suitable means of conjugation may be used.

The mutant igNAR protein-biological conjugate is then injected into a suitable mammal so that it can bind to albumin to form an albumin-mutant igNAR protein-biological complex. The effect of the mutant igNAR protein on the half-life of the biological molecule in vivo can then be measured by comparing the half-life of the biological in the albumin-mutant igNAR protein-biological complex with that of the free biological in a parallel control experiment.

Conjugating the biological to the albumin-binding igNAR protein and then allowing the conjugate to bind albumin is found to extend the in vivo half-life of the biological molecule compared to that of free biological.

1. A modified immunoglobulin New Antigen Receptor (igNAR) peptide sequence derived from a wild-type igNAR peptide sequence, which is diversified by mutating the amino acid sequence at 50% or more of the amino acids in the CDR3 loop region, wherein the modified igNAR peptide sequence demonstrates a binding affinity for a target peptide sequence.

2. The modified igNAR peptide of claim 1, which is further diversified by mutating the amino acid sequence at 50% or more of the amino acids in the CDR1 loop region.

3. The modified igNAR peptide of claim 1, which is derived from the variable domain of the Wobbegong shark igNAR peptide (SEQ ID NO: 86).

4. The modified igNAR peptide of claim 1, wherein the modified igNAR peptide sequence contains diversifications at 50% or more of the residues selected from one or more of the group consisting of: SEQ ID NO: 87 and SEQ ID NO: 11.

5. The modified igNAR peptide of claim 1, which comprises a sequence having at least 90% identity in the region of amino acid positions 1 to 84 and 101 to 110 to a sequence selected from one or more of the group consisting of: SEQ ID NO: 8; SEQ ID NO: 10; and SEQ ID NO: 86.

6. The modified igNAR peptide of claim 1, which comprises a sequence having at least 95% identity to SEQ ID NO: 86 in the region of positions 1 to 18, 26 to 84 and 98 to 107.

7. The modified igNAR peptide of claim 1, wherein the CDR3 loop has at least 11 amino acids residues.

8. The modified igNAR peptide of claim 1, wherein the CDR3 loop has 16 amino acids and does not include a cysteine at position 88 of SEQ ID NO: 86.

9. (canceled)

10. The modified igNAR peptide of claim 1, which comprises:
(i) the modified CDR3 sequence of SEQ ID NO: 9; and
(ii) a modified CDR1 sequence selected from one or more of the group consisting of: SEQ ID NOs: 16 to 50.

11. A modified igNAR peptide which comprises at least 70 consecutive amino acids of the peptide of claim 1.

12. The modified igNAR peptide sequence of claim 1, which comprises an amino acid sequence selected from any of the group consisting of SEQ ID NO: 8; 10; 51 to 85; and a sequence having at least 98% identity thereto over a length of at least 100 amino acids.

13. The modified igNAR peptide sequence of claim 1, wherein the target peptide comprises an albumin peptide sequence.

14. The modified igNAR peptide sequence of claim 13, wherein the albumin peptide sequence comprises one of the group selected from: a human; mouse; and rat albumin protein.

15. The modified igNAR peptide sequence of claim 13, which binds the albumin peptide sequence with a dissociation constant (Kd) of less than 10 μM.

16.-27. (canceled)

28. A method of treating, preventing or alleviating a disease in a mammal, the method comprising administering to a subject in need thereof a therapeutically effective amount of the modified igNAR peptide of claim 1.

29. A pharmaceutical composition comprising the peptide of claim 1.

30. A method of extending the in vivo or serum half-life of a therapeutic molecule by conjugating or associating the therapeutic molecule with a modified igNAR peptide sequence capable of binding to an albumin.

31. The method of claim 30, wherein the igNAR peptide sequence comprises a peptide sequence derived from a wild-type igNAR peptide sequence, which is diversified by mutating the amino acid sequence at 50% or more of the amino acids in the CDR3 loop region, wherein the modified igNAR peptide sequence demonstrates a binding affinity for a target peptide sequence.

32. (canceled)

33. The method of claim 30, wherein the albumin is a human serum albumin.


SEQUENCE LISTING (96 sequences)

SEQ ID NO: 1 - DNA, 72 bp, Artificial Sequence
  Description of Artificial Sequence Synthetic PCR primer
tacggtgccagctccctcmnnmnnmnnmnnmnnmnnmnngcamnnmnnmnntgctttacacttatacgtgcc

SEQ ID NO: 2 - DNA, 78 bp, Artificial Sequence
  Description of Artificial Sequence Synthetic PCR primer
tacggtgccagctccctcmnnmnnmnnmnnmnnmnnmnnmnnmnngcamnnmnnmnntgctttacacttatacgtgcc

SEQ ID NO: 3 - DNA, 87 bp, Artificial Sequence
  Description of Artificial Sequence Synthetic PCR primer
tacggtgccagctccctcmnnmnnmnnmnnmnnmnnmnnmnnmnnmnnmnnmnnmnnmnnmnnmnntgctttacacttatacgtgcc

SEQ ID NO: 4 - DNA, 93 bp, Artificial Sequence
  Description of Artificial Sequence Synthetic PCR primer
tacggtgccagctccctcmnnmnnmnnmnnmnnmnnmnnmnnmnnmnnmnnmnnmnngcamnnmnnmnnmnntgctttacacttatacgtgcc

SEQ ID NO: 5 - DNA, 26 bp, Artificial Sequence
  Description of Artificial Sequence Synthetic PCR primer
ggccgtcaaggccacgtgtcttgtcc

SEQ ID NO: 6 - DNA, 86 bp, Artificial Sequence
  Description of Artificial Sequence Synthetic PCR primer
aaaaaagccatggcaagggtggaccaaacaccaagaatagcaacaaaagagacgggcgaatcactgaccatcaattgcgtcctaag

SEQ ID NO: 7 - DNA, 54 bp, Artificial Sequence
  Description of Artificial Sequence Synthetic PCR primer
taggccaattgcggccgcacctcctttcacggttaatacggtgccagctccctc

SEQ ID NO: 8 - protein, 110 aa, Artificial Sequence
  Description of Artificial Sequence Synthetic albumin binding mutant igNAR peptide
RVDQTPRIATKETGESLTINCVLRDTACALDSTNWYRTKLGSTKEQTISIGGRYSETVDEGSNSASLTIRDLRVEDSGTYKCKAAITPFDNWYECLGTRAEGAGTVLTVK

SEQ ID NO: 9 - protein, 16 aa, Artificial Sequence
  Description of Artificial Sequence Synthetic mutated CDR3 loop peptide
AITPFDNWYECLGTRA

SEQ ID NO: 10 - protein, 110 aa, Artificial Sequence
  Description of Artificial Sequence Synthetic albumin binding mutant igNAR peptide
RVDQTPRIATKETGESLTINCVLRDTACALDSTNWYRTKLGSTKEQTISIGGRYSETADEGSNPASLTIRDLRVEDSGTYKCKAAITPFDNWYECLGTRAEGAGTVLTVK

SEQ ID NO: 11 - protein, 7 aa, Orectolobus sp.
INCVLRD

SEQ ID NO: 12 - DNA, 72 bp, Artificial Sequence
  Description of Artificial Sequence Synthetic PCR primer
gaccatcaattgcgtcctaagagatnnknnktgtnnknnknnknnkacgaattggtatcggacaaaattggg

SEQ ID NO: 13 - DNA, 25 bp, Artificial Sequence
  Description of Artificial Sequence Synthetic PCR primer
caactttcaacagtttcagcagagg

SEQ ID NO: 14 - DNA, 86 bp, Artificial Sequence
  Description of Artificial Sequence Synthetic PCR primer
agaatttccatggcactcgtggaccaaacaccaagaatagcaacaaaagagacgggcgaatcactgaccatcaattgcgtcctaag

SEQ ID NO: 15 - DNA, 54 bp, Artificial Sequence; identical to SEQ ID NO: 7
  Description of Artificial Sequence Synthetic PCR primer

SEQ ID NO: 16 - protein, 7 aa, Artificial Sequence
  Description of Artificial Sequence Synthetic mutated CDR1 loop peptide
TLCHMSF

SEQ ID NO: 17 - protein, 7 aa, Artificial Sequence
  Description of Artificial Sequence Synthetic mutated CDR1 loop peptide
TICQIST

SEQ ID NO: 18 - protein, 7 aa, Artificial Sequence
  Description of Artificial Sequence Synthetic mutated CDR1 loop peptide
SLCQMHT

SEQ ID NO: 19 - protein, 7 aa, Artificial Sequence
  Description of Artificial Sequence Synthetic mutated CDR1 loop peptide
TICSLAF

SEQ ID NO: 20 - protein, 7 aa, Artificial Sequence
  Description of Artificial Sequence Synthetic mutated CDR1 loop peptide
SLCWMYI

SEQ ID NO: 21 - protein, 7 aa, Artificial Sequence
  Description of Artificial Sequence Synthetic mutated CDR1 loop peptide
TICWQTE

SEQ ID NO: 22 - protein, 7 aa, Artificial Sequence
  Description of Artificial Sequence Synthetic mutated CDR1 loop peptide
SLCWMMD

SEQ ID NO: 23 - protein, 7 aa, Artificial Sequence
  Description of Artificial Sequence Synthetic mutated CDR1 loop peptide
TLCTMIW

SEQ ID NO: 24 - protein, 7 aa, Artificial Sequence
  Description of Artificial Sequence Synthetic mutated CDR1 loop peptide
SMCHMTQ

SEQ ID NO: 25 - protein, 7 aa, Artificial Sequence
  Description of Artificial Sequence Synthetic mutated CDR1 loop peptide
SLCGIHE

SEQ ID NO: 26 - protein, 7 aa, Artificial Sequence
  Description of Artificial Sequence Synthetic mutated CDR1 loop peptide
TICLQEE

SEQ ID NO: 27 - protein, 7 aa, Artificial Sequence
  Description of Artificial Sequence Synthetic mutated CDR1 loop peptide
TLCGAAD

SEQ ID NO: 28 - protein, 7 aa, Artificial Sequence
  Description of Artificial Sequence Synthetic mutated CDR1 loop peptide
TLCRMTG

SEQ ID NO: 29 - protein, 7 aa, Artificial Sequence
  Description of Artificial Sequence Synthetic mutated CDR1 loop peptide
SLCHIKD

SEQ ID NO: 30 - protein, 7 aa, Artificial Sequence
  Description of Artificial Sequence Synthetic mutated CDR1 loop peptide
SMCHMTQ

SEQ ID NO: 31 - protein, 7 aa, Artificial Sequence
  Description of Artificial Sequence Synthetic mutated CDR1 loop peptide
TMCEFQD

SEQ ID NO: 32 - protein, 7 aa, Artificial Sequence
  Description of Artificial Sequence Synthetic mutated CDR1 loop peptide
TFCELAE

SEQ ID NO: 33 - protein, 7 aa, Artificial Sequence
  Description of Artificial Sequence Synthetic mutated CDR1 loop peptide
SMCHLQE

SEQ ID NO: 34 - protein, 7 aa, Artificial Sequence
  Description of Artificial Sequence Synthetic mutated CDR1 loop peptide
TMCHWQD

SEQ ID NO: 35 - protein, 7 aa, Artificial Sequence
  Description of Artificial Sequence Synthetic mutated CDR1 loop peptide
TLCHIAV

SEQ ID NO: 36 - protein, 7 aa, Artificial Sequence
  Description of Artificial Sequence Synthetic mutated CDR1 loop peptide
TLCHMAW

SEQ ID NO: 37 - protein, 7 aa, Artificial Sequence
  Description of Artificial Sequence Synthetic mutated CDR1 loop peptide
TLCHLYS

SEQ ID NO: 38 - protein, 7 aa, Artificial Sequence
  Description of Artificial Sequence Synthetic mutated CDR1 loop peptide
TLCHPAW

SEQ ID NO: 39 - protein, 7 aa, Artificial Sequence
  Description of Artificial Sequence Synthetic mutated CDR1 loop peptide
TLCNIEL

SEQ ID NO: 40 - protein, 7 aa, Artificial Sequence
  Description of Artificial Sequence Synthetic mutated CDR1 loop peptide
SLCGIHE

SEQ ID NO: 41 - protein, 7 aa, Artificial Sequence
  Description of Artificial Sequence Synthetic mutated CDR1 loop peptide
TFCILHD

SEQ ID NO: 42 - protein, 7 aa, Artificial Sequence
  Description of Artificial Sequence Synthetic mutated CDR1 loop peptide
TACALDS

SEQ ID NO: 43 - protein, 7 aa, Artificial Sequence
  Description of Artificial Sequence Synthetic mutated CDR1 loop peptide
SMCWAII

SEQ ID NO: 44 - protein, 7 aa, Artificial Sequence
  Description of Artificial Sequence Synthetic mutated CDR1 loop peptide
TLCVVPQ

SEQ ID NO: 45 - protein, 7 aa, Artificial Sequence
  Description of Artificial Sequence Synthetic mutated CDR1 loop peptide
TMCLFMV

SEQ ID NO: 46 - protein, 7 aa, Artificial Sequence
  Description of Artificial Sequence Synthetic mutated CDR1 loop peptide
TLCDLMI

SEQ ID NO: 47 - protein, 7 aa, Artificial Sequence
  Description of Artificial Sequence Synthetic mutated CDR1 loop peptide
TICLQEE

SEQ ID NO: 48 - protein, 7 aa, Artificial Sequence
  Description of Artificial Sequence Synthetic mutated CDR1 loop peptide
TLCGAAD

SEQ ID NO: 49 - protein, 7 aa, Artificial Sequence
  Description of Artificial Sequence Synthetic mutated CDR1 loop peptide
SLCHISF

SEQ ID NO: 50 - protein, 7 aa, Artificial Sequence
  Description of Artificial Sequence Synthetic mutated CDR1 loop peptide
TLCIMTS

SEQ ID NO: 51 - protein, 110 aa, Artificial Sequence
  Description of Artificial Sequence Synthetic albumin binding mutant igNAR polypeptide
RVDQTPRIATKETGESLTTLCHMSFTACALDSTNWYRTKLGSTKEQTISIGGRYSETADEGSNPASLTIRDLRVEDSGTYKCKAAITPFDNWYECLGTRAEGAGTVLTVK

SEQ ID NO: 52 - protein, 110 aa, Artificial Sequence
  Description of Artificial Sequence Synthetic albumin binding mutant igNAR polypeptide
RVDQTPRIATKETGESLTTICQISTTACALDSTNWYRTKLGSTKEQTISIGGRYSETADEGSNPASLTIRDLRVEDSGTYKCKAAITPFDNWYECLGTRAEGAGTVLTVK

SEQ ID NO: 53 - protein, 110 aa, Artificial Sequence
  Description of Artificial Sequence Synthetic albumin binding mutant igNAR polypeptide
RVDQTPRIATKETGESLTSLCQMHTTACALDSTNWYRTKLGSTKEQTISIGGRYSETADEGSNPASLTIRDLRVEDSGTYKCKAAITPFDNWYECLGTRAEGAGTVLTVK

SEQ ID NO: 54 - protein, 110 aa, Artificial Sequence
  Description of Artificial Sequence Synthetic albumin binding mutant igNAR polypeptide
RVDQTPRIATKETGESLTTICSLAFTACALDSTNWYRTKLGSTKEQTISIGGRYSETADEGSNPASLTIRDLRVEDSGTYKCKAAITPFDNWYECLGTRAEGAGTVLTVK

SEQ ID NO: 55 - protein, 110 aa, Artificial Sequence
  Description of Artificial Sequence Synthetic albumin binding mutant igNAR polypeptide
RVDQTPRIATKETGESLTSLCWMYITACALDSTNWYRTKLGSTKEQTISIGGRYSETADEGSNPASLTIRDLRVEDSGTYKCKAAITPFDNWYECLGTRAEGAGTVLTVK

SEQ ID NO: 56 - protein, 110 aa, Artificial Sequence
  Description of Artificial Sequence Synthetic albumin binding mutant igNAR polypeptide
RVDQTPRIATKETGESLTTICWQTETACALDSTNWYRTKLGSTKEQTISIGGRYSETADEGSNPASLTIRDLRVEDSGTYKCKAAITPFDNWYECLGTRAEGAGTVLTVK

SEQ ID NO: 57 - protein, 110 aa, Artificial Sequence
  Description of Artificial Sequence Synthetic albumin binding mutant igNAR polypeptide
RVDQTPRIATKETGESLTSLCWMMDTACALDSTNWYRTKLGSTKEQTISIGGRYSETADEGSNPASLTIRDLRVEDSGTYKCKAAITPFDNWYECLGTRAEGAGTVLTVK

SEQ ID NO: 58 - protein, 110 aa, Artificial Sequence
  Description of Artificial Sequence Synthetic albumin binding mutant igNAR polypeptide
RVDQTPRIATKETGESLTTLCTMIWTACALDSTNWYRTKLGSTKEQTISIGGRYSETADEGSNPASLTIRDLRVEDSGTYKCKAAITPFDNWYECLGTRAEGAGTVLTVK

SEQ ID NO: 59 - protein, 110 aa, Artificial Sequence
  Description of Artificial Sequence Synthetic albumin binding mutant igNAR polypeptide
RVDQTPRIATKETGESLTSMCHMTQTACALDSTNWYRTKLGSTKEQTISIGGRYSETADEGSNPASLTIRDLRVEDSGTYKCKAAITPFDNWYECLGTRAEGAGTVLTVK

SEQ ID NO: 60 - protein, 110 aa, Artificial Sequence
  Description of Artificial Sequence Synthetic albumin binding mutant igNAR polypeptide
RVDQTPRIATKETGESLTSLCGIHETACALDSTNWYRTKLGSTKEQTISIGGRYSETADEGSNPASLTIRDLRVEDSGTYKCKAAITPFDNWYECLGTRAEGAGTVLTVK

SEQ ID NO: 61 - protein, 110 aa, Artificial Sequence
  Description of Artificial Sequence Synthetic albumin binding mutant igNAR polypeptide
RVDQTPRIATKETGESLTTICLQEETACALDSTNWYRTKLGSTKEQTISIGGRYSETADEGSNPASLTIRDLRVEDSGTYKCKAAITPFDNWYECLGTRAEGAGTVLTVK

SEQ ID NO: 62 - protein, 110 aa, Artificial Sequence
  Description of Artificial Sequence Synthetic albumin binding mutant igNAR polypeptide
RVDQTPRIATKETGESLTTLCGAADTACALDSTNWYRTKLGSTKEQTISIGGRYSETADEGSNPASLTIRDLRVEDSGTYKCKAAITPFDNWYECLGTRAEGAGTVLTVK

SEQ ID NO: 63 - protein, 110 aa, Artificial Sequence
  Description of Artificial Sequence Synthetic albumin binding mutant igNAR polypeptide
RVDQTPRIATKETGESLTTLCRMTGTACALDSTNWYRTKLGSTKEQTISIGGRYSETADEGSNPASLTIRDLRVEDSGTYKCKAAITPFDNWYECLGTRAEGAGTVLTVK

SEQ ID NO: 64 - protein, 110 aa, Artificial Sequence
  Description of Artificial Sequence Synthetic albumin binding mutant igNAR polypeptide
RVDQTPRIATKETGESLTSLCHIKDTACALDSTNWYRTKLGSTKEQTISIGGRYSETADEGSNPASLTIRDLRVEDSGTYKCKAAITPFDNWYECLGTRAEGAGTVLTVK

SEQ ID NO: 65 - protein, 110 aa, Artificial Sequence; identical to SEQ ID NO: 59
  Description of Artificial Sequence Synthetic albumin binding mutant igNAR polypeptide

SEQ ID NO: 66 - protein, 110 aa, Artificial Sequence
  Description of Artificial Sequence Synthetic albumin binding mutant igNAR polypeptide
RVDQTPRIATKETGESLTTMCEFQDTACALDSTNWYRTKLGSTKEQTISIGGRYSETADEGSNPASLTIRDLRVEDSGTYKCKAAITPFDNWYECLGTRAEGAGTVLTVK

SEQ ID NO: 67 - protein, 110 aa, Artificial Sequence
  Description of Artificial Sequence Synthetic albumin binding mutant igNAR polypeptide
RVDQTPRIATKETGESLTTFCELAETACALDSTNWYRTKLGSTKEQTISIGGRYSETADEGSNPASLTIRDLRVEDSGTYKCKAAITPFDNWYECLGTRAEGAGTVLTVK

SEQ ID NO: 68 - protein, 110 aa, Artificial Sequence
  Description of Artificial Sequence Synthetic albumin binding mutant igNAR polypeptide
RVDQTPRIATKETGESLTSMCHLQETACALDSTNWYRTKLGSTKEQTISIGGRYSETADEGSNPASLTIRDLRVEDSGTYKCKAAITPFDNWYECLGTRAEGAGTVLTVK

SEQ ID NO: 69 - protein, 110 aa, Artificial Sequence
  Description of Artificial Sequence Synthetic albumin binding mutant igNAR polypeptide
RVDQTPRIATKETGESLTTMCHWQDTACALDSTNWYRTKLGSTKEQTISIGGRYSETADEGSNPASLTIRDLRVEDSGTYKCKAAITPFDNWYECLGTRAEGAGTVLTVK

SEQ ID NO: 70 - protein, 110 aa, Artificial Sequence
  Description of Artificial Sequence Synthetic albumin binding mutant igNAR polypeptide
RVDQTPRIATKETGESLTTLCHIAVTACALDSTNWYRTKLGSTKEQTISIGGRYSETADEGSNPASLTIRDLRVEDSGTYKCKAAITPFDNWYECLGTRAEGAGTVLTVK

SEQ ID NO: 71 - protein, 110 aa, Artificial Sequence
  Description of Artificial Sequence Synthetic albumin binding mutant igNAR polypeptide
RVDQTPRIATKETGESLTTLCHMAWTACALDSTNWYRTKLGSTKEQTISIGGRYSETADEGSNPASLTIRDLRVEDSGTYKCKAAITPFDNWYECLGTRAEGAGTVLTVK

SEQ ID NO: 72 - protein, 110 aa, Artificial Sequence
  Description of Artificial Sequence Synthetic albumin binding mutant igNAR polypeptide
RVDQTPRIATKETGESLTTLCHLYSTACALDSTNWYRTKLGSTKEQTISIGGRYSETADEGSNPASLTIRDLRVEDSGTYKCKAAITPFDNWYECLGTRAEGAGTVLTVK

SEQ ID NO: 73 - protein, 110 aa, Artificial Sequence
  Description of Artificial Sequence Synthetic albumin binding mutant igNAR polypeptide
RVDQTPRIATKETGESLTTLCHPAWTACALDSTNWYRTKLGSTKEQTISIGGRYSETADEGSNPASLTIRDLRVEDSGTYKCKAAITPFDNWYECLGTRAEGAGTVLTVK

SEQ ID NO: 74 - protein, 110 aa, Artificial Sequence
  Description of Artificial Sequence Synthetic albumin binding mutant igNAR polypeptide
RVDQTPRIATKETGESLTTLCNIELTACALDSTNWYRTKLGSTKEQTISIGGRYSETADEGSNPASLTIRDLRVEDSGTYKCKAAITPFDNWYECLGTRAEGAGTVLTVK

SEQ ID NO: 75 - protein, 110 aa, Artificial Sequence; identical to SEQ ID NO: 60
  Description of Artificial Sequence Synthetic albumin binding mutant igNAR polypeptide

SEQ ID NO: 76 - protein, 110 aa, Artificial Sequence
  Description of Artificial Sequence Synthetic albumin binding mutant igNAR polypeptide
RVDQTPRIATKETGESLTTFCILHDTACALDSTNWYRTKLGSTKEQTISIGGRYSETADEGSNPASLTIRDLRVEDSGTYKCKAAITPFDNWYECLGTRAEGAGTVLTVK

SEQ ID NO: 77 - protein, 110 aa, Artificial Sequence
  Description of Artificial Sequence Synthetic albumin binding mutant igNAR polypeptide
RVDQTPRIATKETGESLTTACALDSTACALDSTNWYRTKLGSTKEQTISIGGRYSETADEGSNPASLTIRDLRVEDSGTYKCKAAITPFDNWYECLGTRAEGAGTVLTVK

SEQ ID NO: 78 - protein, 110 aa, Artificial Sequence
  Description of Artificial Sequence Synthetic albumin binding mutant igNAR polypeptide
RVDQTPRIATKETGESLTSMCWAIITACALDSTNWYRTKLGSTKEQTISIGGRYSETADEGSNPASLTIRDLRVEDSGTYKCKAAITPFDNWYECLGTRAEGAGTVLTVK

SEQ ID NO: 79 - protein, 110 aa, Artificial Sequence
  Description of Artificial Sequence Synthetic albumin binding mutant igNAR polypeptide
RVDQTPRIATKETGESLTTLCVVPQTACALDSTNWYRTKLGSTKEQTISIGGRYSETADEGSNPASLTIRDLRVEDSGTYKCKAAITPFDNWYECLGTRAEGAGTVLTVK

SEQ ID NO: 80 - protein, 110 aa, Artificial Sequence
  Description of Artificial Sequence Synthetic albumin binding mutant igNAR polypeptide
RVDQTPRIATKETGESLTTMCLFMVTACALDSTNWYRTKLGSTKEQTISIGGRYSETADEGSNPASLTIRDLRVEDSGTYKCKAAITPFDNWYECLGTRAEGAGTVLTVK

SEQ ID NO: 81 - protein, 110 aa, Artificial Sequence
  Description of Artificial Sequence Synthetic albumin binding mutant igNAR polypeptide
RVDQTPRIATKETGESLTTLCDLMITACALDSTNWYRTKLGSTKEQTISIGGRYSETADEGSNPASLTIRDLRVEDSGTYKCKAAITPFDNWYECLGTRAEGAGTVLTVK

SEQ ID NO: 82 - protein, 110 aa, Artificial Sequence; identical to SEQ ID NO: 61
  Description of Artificial Sequence Synthetic albumin binding mutant igNAR polypeptide

SEQ ID NO: 83 - protein, 110 aa, Artificial Sequence; identical to SEQ ID NO: 62
  Description of Artificial Sequence Synthetic albumin binding mutant igNAR polypeptide

SEQ ID NO: 84 - protein, 110 aa, Artificial Sequence
  Description of Artificial Sequence Synthetic albumin binding mutant igNAR polypeptide
RVDQTPRIATKETGESLTSLCHISFTACALDSTNWYRTKLGSTKEQTISIGGRYSETADEGSNPASLTIRDLRVEDSGTYKCKAAITPFDNWYECLGTRAEGAGTVLTVK

SEQ ID NO: 85 - protein, 110 aa, Artificial Sequence
  Description of Artificial Sequence Synthetic albumin binding mutant igNAR polypeptide
RVDQTPRIATKETGESLTTLCIMTSTACALDSTNWYRTKLGSTKEQTISIGGRYSETADEGSNPASLTIRDLRVEDSGTYKCKAAITPFDNWYECLGTRAEGAGTVLTVK

SEQ ID NO: 86 - protein, 107 aa, Orectolobus sp.
RVDQTPRIATKETGESLTINCVLRDTACALDSTNWYRTKLGSTKEQTISIGGRYSETVDEGSNSASLTIRDLRVEDSGTYKCKAYRRCAFNTGVGYKEGAGTVLTVK

SEQ ID NO: 87 - protein, 13 aa, Orectolobus sp.
YRRCAFNTGVGYK

SEQ ID NO: 88 - protein, 124 aa, Artificial Sequence
  Description of Artificial Sequence Synthetic naive igNAR polypeptide library
RVDQTPRIATKETGESLTINCVLRDTACALDSTNWYRTKLGSTKEQTISIGGRYSETVDEGSNSASLTIRDLRVEDSGTYKCKAXXXXXXXXXXXXXXXXXXXXXXXXXXXXXXEGAGTVLTVK

SEQ ID NO: 89 - protein, 105 aa, Artificial Sequence
  Description of Artificial Sequence Synthetic naive igNAR polypeptide library
RVDQTPRIATKETGESLTINCVLRDTACALDSTNWYRTKLGSTKEQTISIGGRYSETVDEGSNSASLTIRDLRVEDSGTYKCKAXXXCXXXXXXXEGAGTVLTVK

SEQ ID NO: 90 - protein, 107 aa, Artificial Sequence
  Description of Artificial Sequence Synthetic naive igNAR polypeptide library
RVDQTPRIATKETGESLTINCVLRDTACALDSTNWYRTKLGSTKEQTISIGGRYSETVDEGSNSASLTIRDLRVEDSGTYKCKAXXXCXXXXXXXXXEGAGTVLTVK

SEQ ID NO: 91 - protein, 110 aa, Artificial Sequence
  Description of Artificial Sequence Synthetic naive igNAR polypeptide library
RVDQTPRIATKETGESLTINCVLRDTACALDSTNWYRTKLGSTKEQTISIGGRYSETVDEGSNSASLTIRDLRVEDSGTYKCKAXXXXXXXXXXXXXXXXEGAGTVLTVK

SEQ ID NO: 92 - protein, 112 aa, Artificial Sequence
  Description of Artificial Sequence Synthetic naive igNAR polypeptide library
RVDQTPRIATKETGESLTINCVLRDTACALDSTNWYRTKLGSTKEQTISIGGRYSETVDEGSNSASLTIRDLRVEDSGTYKCKAXXXXCXXXXXXXXXXXXXEGAGTVLTVK

SEQ ID NO: 93 - DNA, 321 bp, Orectolobus sp.
  CDS (1)..(321)
agggtggaccaaacaccaagaatagcaacaaaagagacgggcgaatcactgaccatcaattgcgtcctaagagatactgcgtgtgcattagacagtacgaattggtatcggacaaaattgggttcaacaaaggagcagacaatatcaattggcggacgatatagtgaaacagtcgacgaaggatcaaactctgcttctctgacaattcgtgatctgagagttgaagacagtggcacgtataagtgtaaagcatataggagatgcgcctttaatactggagtgggatacaaggagggagctggcaccgtattaaccgtgaaa

SEQ ID NO: 94 - DNA, 184 bp, Artificial Sequence
  Description of Artificial Sequence Synthetic polynucleotide
aacagctatgaccatgattacgccaagcttgcatgcaaattctatttcaaggagacagtcataaatgaaatacctattgcctacggcagccgctggattgttattactcgcggcccagccggccatggccgaggtgcaactgcagtaataggcggccgcataggggggaggagggtcctctgct

SEQ ID NO: 95 - protein, 22 aa, Artificial Sequence
  Description of Artificial Sequence Synthetic peptide
MKYLLPTAAAGLLLLAAQPAMA

SEQ ID NO: 96 - protein, 7 aa, Artificial Sequence
  Description of Artificial Sequence Synthetic peptide
GGGGSSA